(19) United States
(12) Patent Application Publication
(10) Pub. No.: US 2026/0116945 A1
(43) Pub. Date: Apr. 30, 2026

(54) ENGINEERED PROTEASOME AND USES THEREOF

(71) Applicant: SEOUL NATIONAL UNIVERSITY R&DB FOUNDATION, Seoul (KR)

(72) Inventors: Min Jae LEE, Seoul (KR); Seh Hoon PARK, Seoul (KR); Yejin JANG, Seoul (KR)

(21) Appl. No.: 19/011,082

(22) Filed: Jan. 6, 2025

(30) Foreign Application Priority Data
Apr. 17, 2024 (KR) 10-2024-0051225

(51) Int. Cl. C07K 14/705; C07K 16/00; C12N 15/62

(52) U.S. Cl. CPC C07K 14/705; C07K 16/00; C12N 15/62; C07K 2319/01; C07K 2319/70

(57) ABSTRACT

Disclosed are a fusion protein comprising a ubiquitin receptor and uses thereof. Specifically, disclosed are a targeted protein degradation technology that directly uses the proteasome, a final organelle that performs protein degradation, as degradation machinery for pathogenic proteins, as well as a fusion protein comprising a ubiquitin receptor, which is a ubiquitin recognition subunit of a proteasome regulatory particle, and uses thereof.

### CROSS-REFERENCE TO RELATED APPLICATION

The priority of Republic of Korea Patent Application 10-2024-0051225 filed Apr. 17, 2024 is hereby claimed under 35 USC § 119. The disclosure of Republic of Korea Patent Application 10-2024-0051225 is hereby incorporated herein by reference, in its entirety, for all purposes.

### SEQUENCE LISTING

This application includes an electronically submitted sequence listing in .xml format. The .xml file contains a sequence listing entitled “754_SeqListing.xml” created on Jan. 6, 2025 and is 35, 719 bytes in size. The sequence listing contained in this .xml file is part of the specification and is hereby incorporated by reference herein in its entirety.

### BACKGROUND OF THE INVENTION

### Field of the Invention

The present invention relates to an engineered proteasome, for example, a fusion protein comprising a ubiquitin receptor and a target-targeting moiety, and uses thereof. Specifically, the present invention relates to a targeted protein degradation technology that directly uses the proteasome, a final organelle that performs protein degradation, as degradation machinery for pathogenic proteins, and to a fusion protein comprising a ubiquitin receptor, which is a ubiquitin recognition subunit of a proteasome regulatory particle, and a target-targeting moiety, and uses thereof.

### Description of the Related Art

A significant portion of the cell signaling process is regulated through post-translational modifications such as protein phosphorylation or degradation. Protein phosphorylation is a reversible process involving phosphorylation and dephosphorylation enzymes, and thus a specific protein can exist in two states depending on the signal applied to the cell, but protein degradation is an irreversible process.

The ubiquitin-proteasome system (UPS) is an important mechanism for protein degradation in the cytoplasm.

Proteasomes not only function to maintain cellular homeostasis by removing damaged or unnecessary proteins, but also participate in cell cycle processes or transcriptional regulation processes that should be precisely regulated over time. During the cell cycle, degradation of cyclin proteins by the proteasome induces the transition from anaphase to metaphase. Degradation of proteins induces the transition from anaphase to metaphase. In addition, the proteasome precisely regulates the transcription process by degrading short-lived transcription factors such as c-fos, c-myc, and c-jun. Furthermore, it regulates apoptosis, differentiation, and DNA damage repair processes, and also participates in the process of antigen peptide formation by MHC class I molecules.

Ubiquitin is a peptide consisting of 76 amino acids and has a globular structure. It has a very high degree of similarity between species. The ubiquitination process of proteins involves three types of enzymes: E1, E2, and E3. E1 enzymes are ubiquitin-activating enzymes that activate ubiquitin by forming a thiol ester intermediate with ubiquitin. During this process, ATP is consumed. Activated ubiquitin is transferred to the ubiquitin-conjugating enzyme E2, and ubiquitin attached to E2 is bound to a substrate protein by the ubiquitin ligase E3.

The ubiquitination process of proteins consists of several steps. The ubiquitin activation step involves E1 enzymes, but in the subsequent steps, substrate-specific ubiquitination is carried out by combinations between various types of E2 and E3 enzymes.

In general, the E3 group is important in the combination of E2, E3, and substrate complexes and in the regulation of ubiquitination. E3 enzymes are broadly divided into three groups: RING-finger E3, HECT (homologous to E6-AP COOH-terminus) domain E3, and U-box E3. The three groups have unique E2 binding domains, called RING-finger, HECT and U-box domains, respectively, and further have domains that bind substrate proteins. E3 enzymes function to bring substrate proteins into close proximity with E2. Since there is a domain within E3 that determines the substrate protein, the activity of a specific substrate protein may be controlled by regulating a specific E3.

Proteins tagged with ubiquitin are degraded by the 26S proteasome, an ATP-dependent protease complex.

Proteolysis targeting chimera (PROTAC) has been considered as a technology that degrades target proteins by utilizing this protein degradation mechanism.

PROTACs typically use bifunctional molecules that can simultaneously bind to E3 and target proteins, including E3 enzyme ligands and target protein ligands, and induce in vivo degradation of disease-causing target proteins through ubiquitination (“Targeted protein degradation by PROTACs”, T. K. Neklesa, J. D. Winkler, C. M. Crews, Pharmacology & Therapeutics, 2017, 174, 138.).

Under this technical background, the inventors of the present application have developed a platform technology for targeted protein degradation independent of ubiquitination through a fusion protein comprising a ubiquitin receptor, which is a ubiquitin recognition subunit of a proteasome regulatory particle, thereby completing the present invention.

### SUMMARY OF THE INVENTION

An object of the present invention is to provide an engineered proteasome for delivering a target protein directly to the proteasome.

Another object of the present invention is to provide a fusion protein comprising a ubiquitin receptor and a target-targeting moiety.

Still another object of the present invention is to provide a composition for degrading a target protein comprising the fusion protein.

Yet another object of the present invention is to provide an isolated nucleic acid encoding the fusion protein.

Still yet another object of the present invention is to provide an expression vector comprising the nucleic acid.

A further object of the present invention is to provide an isolated host cell comprising the expression vector.

To achieve the above objects, the present invention relates to a fusion protein comprising a ubiquitin receptor and a target-targeting moiety conjugated thereto, wherein the target-targeting moiety is conjugated to the ubiquitin receptor which is a ubiquitin recognition subunit of a proteasome regulatory particle.

The ubiquitin receptor may bind to a proteasome. The fusion protein according to the present invention may deliver a target protein directly to the proteasome.

The ubiquitin receptor may be selected from the group consisting of PSMD2 (Proteasome 26S Subunit Ubiquitin Receptor, Non-ATPase 2), PSMD4 (Proteasome 26S Subunit, Non-ATPase 4), and ADRM1 (Adhesion-regulating molecule 1).

The target-targeting moiety may be an antibody or an antigen-binding fragment thereof.

The antibody or antigen-binding fragment thereof may be a single-chain variable fragment (scFv) or a nanobody.

The target-targeting moiety may be conjugated to the N- or C-terminus of the ubiquitin receptor.

The target-targeting moiety may be conjugated directly or via a linker to the ubiquitin receptor.

The linker may comprise a rigid linker or a flexible linker.

The present invention also relates to a composition for degrading a target protein comprising the fusion protein.

The composition may degrade the target protein by inducing binding of a proteasome to the target protein bound to the fusion protein.

The present invention also relates to an isolated nucleic acid encoding the fusion protein.

The present invention also relates to an expression vector containing the nucleic acid.

The present invention also relates to an isolated host cell comprising the expression vector.

According to the present invention, a recombinant fusion protein in which a target-targeting moiety is conjugated to a ubiquitin recognition subunit of a proteasome regulatory particle may be assembled into an endogenous proteasome to induce an engineered proteasome, and the formed engineered proteasome may induce rapid target protein degradation. It was confirmed that such degradation is dependent on the proteasome and independent of ubiquitin.

According to the present invention, it is possible to target and degrade a pathogenic protein as a target protein, and thus universal application to diseases is possible.

### BRIEF DESCRIPTION OF THE DRAWINGS

FIG. 1 shows gradual degradation of a target protein (c-Fos) by dose-dependent expression of a recombinant protein (scFv(c-Fos)-ADRM1). Specifically, FIG. 1 in A therein shows the results of immunoblotting after overexpression (co-transfection) of the recombinant protein (dose dependent) and the target protein in a mammalian cell line (A549), FIG. 1 in B therein shows the results of quantifying the blot of the target protein by independently repeating the experiment of A of FIG. 1 three times, and FIG. 1 in C therein shows the results of analyzing the mRNA level of the target protein by qPCR.

FIG. 2 shows gradual degradation of a target protein (c-Fos) by dose-dependent expression of a recombinant protein (ADRM1-scFv(c-Fos)). Specifically, FIG. 2 in A therein shows the results of immunoblotting after overexpression (co-transfection) of the recombinant protein and the target protein (dose dependent) in a mammalian cell line (A549), FIG. 2 in B therein shows the results of quantifying the blot of the target protein by independently repeating the experiment of A of FIG. 2 three times, and FIG. 2 in C therein shows the results of analyzing the mRNA level of the target protein by qPCR.

FIG. 3 shows changes in the stability of a target protein upon expression of a recombinant protein (ADRM1-related). Specifically, FIG. 3 in A therein shows the results of performing immunoblotting after overexpression of the recombinant protein (scFv(c-Fos)-ADRM1) followed by treatment with cycloheximide (CHX), FIG. 3 in B therein is a graph quantitatively showing the change in target protein (c-Fos)/total protein in the experiment of A of FIG. 3, FIG. 3 in C therein shows the results of performing immunoblotting after overexpression of the recombinant protein (ADRM1-scFv(c-Fos)) followed by treatment with cycloheximide (CHX), and FIG. 3 in D therein is a graph quantitatively showing the change in target protein (c-Fos)/total protein in the experiment of C of FIG. 3.

FIG. 4 shows degradation of other target proteins by the ProMeD system. Specifically, FIG. 4 in A therein shows the results of performing a degradation assay targeting HA-tau after conjugating another antibody (scFv(HA)) that binds to HA to the N-terminus of ADRM1, and FIG. 4 in B therein shows the results of performing a degradation assay targeting GFP-ODC after conjugating an antibody (VHH(GFP)) that binds GFP to the N-terminus of ADRM1.

FIG. 5 shows gradual degradation of a target protein (c-Fos) by dose-dependent expression of a recombinant protein (PSMD4-scFv(c-Fos)) using a novel ubiquitin recognition subunit. Specifically, FIG. 5 in A therein shows the results of immunoblotting after overexpression (co-transfection) of the recombinant protein (dose dependent) and the target protein in a mammalian cell line (A549), FIG. 5 in B therein shows the results of quantifying the blot of the target protein by independently repeating the experiment of A of FIG. 5 three times, and FIG. 5 in C therein shows the results of analyzing the mRNA level of the target protein by qPCR.

FIG. 6 shows changes in the stability of a target protein upon expression of a recombinant protein (PSMD4-related). Specifically, FIG. 6 in A therein shows the results of performing immunoblotting after overexpression of the recombinant protein (PSMD4-scFv(c-Fos)) followed by treatment with cycloheximide (CHX), and FIG. 6 in B therein is a graph quantitatively showing the changes in target protein (c-Fos)/total protein in the experiment of A of FIG. 6.

FIG. 7 shows analysis of recombinant protein-dependent degradation of a target protein and the degradation of other target proteins by the recombinant protein using PSMD4. Specifically, FIG. 7 in A therein shows the results of degradation assays performed by adding controls expressing the two parts of the recombinant protein, that is, the ubiquitin subunit (PSMD4-HTB) and the antibody (scFv(c-Fos)), alone, respectively, FIG. 7 in B therein shows the results of performing a degradation assay targeting Flag-TDP43 after conjugating another antibody (scFv(Flag)) that binds to Flag to the C-terminus of PSMD4, and FIG. 7 in C therein shows the results of performing a degradation assay targeting GFP-ODC after conjugating an antibody (VHH(GFP)) that binds GFP to the C-terminus of PSMD4.

FIG. 8 shows the generation of e-proteasome by expression of a recombinant protein. Specifically, FIG. 8 in A therein is a schematic diagram showing e-proteasome formation, FIG. 8 in B therein shows the results of performing immunoblotting of a protein purified after overexpression of the recombinant protein (scFv(c-Fos)-ADRM1) in a mammalian cell line (A549), FIG. 8 in C therein shows the results of performing immunoblotting of a protein purified after overexpression of the recombinant protein (PSMD4-scFv(c-Fos)) in the A549 cell line, and FIG. 8 in D therein shows the results of performing immunoblotting after native-PAGE after purifying the recombinant protein (PSMD4-scFv(c-Fos)).

FIG. 9 shows the pathway of target protein degradation induced by a recombinant protein. Specifically, FIG. 9 in A therein shows the results of performing immunoblotting after overexpression of LacZ-V5 and the recombinant protein (scFv(c-Fos)-ADRM1) in mammalian cells followed by treatment with a proteasome inhibitor (MG132), a lysosomal degradation pathway inhibitor (BafA1), and an E1 enzyme inhibitor (MLN7243), and FIG. 9 in B therein shows the results of performing immunoblotting after overexpression of a control and the recombinant protein (PSMD4-scFv(c-Fos)) in mammalian cells followed by treatment with a proteasome inhibitor (MG132), a lysosomal degradation pathway inhibitor (BafA1), and an E1 enzyme inhibitor (MLN7243).

FIG. 10 demonstrates affinity-dependent degradation by ProMeD. Specifically, FIG. 10 in A therein shows the results of a degradation assay performed to compare the ability to degrade c-Fos between a recombinant protein conjugated with an antibody that binds to c-Fos and a recombinant protein conjugated with an antibody that has no affinity for c-Fos, and FIG. 10 in B therein shows the results of a degradation assay performed to compare degradation ability according to affinity for GFP.

FIG. 11 shows the results of degradation of different pathogenic proteins by the ProMeD system. Specifically, it shows the results of a degradation assay in which a recombinant protein in which an antibody (scFv(BRD4)) that binds to BRD4 is conjugated to the C-terminus of ADRM1 was overexpressed together with HA-BRD4 in a mammalian cell line.

FIG. 12 shows the results of degradation of a proto-oncoprotein (c-Fos) through the ProMeD system. Specifically, FIG. 12 in A therein shows the results of a degradation assay in which LacZ-V5 and scFv(c-Fos)-ADRM1 were each overexpressed in the MCF-7 cell line, and FIG. 12 in B therein shows the results of a degradation assay in which LacZ-V5 and scFv(c-Fos)-ADRM1 were each overexpressed in the HCT116 cell line.

FIG. 13 shows the results of endogenous target protein degradation through ProMeD. Specifically, FIG. 13 in A therein shows the results of immunoblotting performed to compare the levels of c-Fos in MCF-7 stably expressing EGFP and MCF-7 stably expressing scFv(c-Fos)-ADRM1, and FIG. 13 in B therein shows the results of immunoblotting performed to compare the levels of c-Fos in A549 stably expressing EGFP and A549 stably expressing scFv(c-Fos)-ADRM1.

### DETAILED DESCRIPTION OF THE INVENTION

Unless otherwise defined, all technical and scientific terms used in the present specification have the same meanings as commonly understood by those skilled in the art to which the present invention pertains. In general, the nomenclature used in the present specification is well known and commonly used in the art.

The present inventors sought to demonstrate the concept of a targeted protein degradation technology (ProMeD) platform that directly utilizes the proteasome, a final organelle that degrades proteins, as degradation machinery for pathogenic proteins. By gene cloning, the present inventors conjugated a targeting antibody to a ubiquitin recognition subunit of a proteasome regulatory particle, thereby inducing an engineered proteasome (e-proteasome) that delivers a target protein directly to the proteasome. The present inventors confirmed target protein degradation by a recombinant protein expressed in cells, and also observed a decrease in the stability of the target protein. By observing the degradation of different target proteins using various antibodies, the present inventors demonstrated the potential of ProMeD as a platform technology.

It has been found that targeted protein degradation by ProMeD is mediated by an engineered proteasome (e-proteasome) induced within cells and is independent of autophagy. Furthermore, it has been confirmed that the targeted protein degradation technology is ubiquitin-independent. The degradation ability of ProMeD has been shown to be a platform with affinity-based specificity where antibody-substrate binding is essential.

Therefore, in one aspect, the present invention relates to a fusion protein comprising a ubiquitin receptor and a target-targeting moiety conjugated thereto.

The ubiquitin receptor is a ubiquitin recognition subunit of the 19S proteasome, and may be used interchangeably with the ubiquitin recognition subunit in the present specification.

The proteasome is the major protein degradation complex responsible for the degradation of a multitude of cellular proteins in eukaryotic cells. The protein complex, present in both the cytoplasm and the nucleus, catalyzes the ATP-dependent proteolysis of short-lived regulatory proteins, as well as the rapid elimination of damaged and abnormal proteins. The 26S proteasome is a large complex that can be dissociated into two functionally distinct subcomplexes: the 20S core particle (CP), which is the proteolytic component, and the 19S regulatory particle (RP), which is responsible for recognizing and translocating substrates into the 20S CP, where they are degraded.

The 20S CP is a 670 kDa barrel-shaped protein complex made up of four stacked, seven-membered rings (4×7 subunits), two outer α rings and two inner β rings (α₁₋₇β₁₋₇β₁₋₇α₁₋₇). The two matching α rings are situated in the outer rims of the barrel, facing the 19S regulatory complex. The proteolytic active sites are located on the two identical β-rings, which are positioned in the center of the 20S complex. In eukaryotes, the catalytic activities of the proteasomes are confined to only three of the β-subunits. β2 possesses tryptic activity (i.e., cleaving after basic residues); β5 displays chymotryptic activity (i.e., cleaving after hydrophobic residues); and β1 has “caspase-like” or “post-acidic” activity. In all three active β-subunits, proteolytic activity is associated with their N-terminal threonine residue, which acts as a nucleophile in peptide-bond hydrolysis.

The ubiquitin receptor may be selected from the group consisting of, for example, PSMD2 (Proteasome 26S Subunit Ubiquitin Receptor, Non-ATPase 2), PSMD4 (Proteasome 26S Subunit, Non-ATPase 4), and ADRM1 (Adhesion-regulating molecule 1), without being limited thereto.

The ubiquitin receptor may be selected from the group consisting of SEQ ID NOS: 1 to 3, without being limited thereto.

The ubiquitin receptor may specifically be PSMD2, PSMD4 or ADRM1. In this case, the ubiquitin receptor may comprise the sequence of SEQ ID NO: 1, 2 or 3.

[PSMD2]
SEQ ID NO: 1
MEEGGRDKAPVQPQQSPAAAPGGTDEKPSGKERRDAGDKDKEQELSEEDK
QLQDELEMLVERLGEKDTSLYRPALEELRRQIRSSTTSMTSVPKPLKFLR
PHYGKLKEIYENMAPGENKRFAADIISVLAMTMSGERECLKYRLVGSQEE
LASWGHEYVRHLAGEVAKEWQELDDAEKVQREPLLTLVKEIVPYNMAHNA
EHEACDLLMEIEQVDMLEKDIDENAYAKVCLYLTSCVNYVPEPENSALLR
CALGVFRKFSRFPEALRLALMLNDMELVEDIFTSCKDVVVQKQMAFMLGR
HGVFLELSEDVEEYEDLTEIMSNVQLNSNFLALARELDIMEPKVPDDIYK
THLENNRFGGSGSQVDSARMNLASSFVNGFVNAAFGQDKLLTDDGNKWLY
KNKDHGMLSAAASLGMILLWDVDGGLTQIDKYLYSSEDYIKSGALLACGI
VNSGVRNECDPALALLSDYVLHNSNTMRLGSIFGLGLAYAGSNREDVLTL
LLPVMGDSKSSMEVAGVTALACGMIAVGSCNGDVTSTILQTIMEKSETEL
KDTYARWLPLGLGLNHLGKGEAIEAILAALEVVSEPFRSFANTLVDVCAY
AGSGNVLKVQQLLHICSEHFDSKEKEEDKDKKEKKDKDKKEAPADMGAHQ
GVAVLGIALIAMGEEIGAEMALRTFGHLLRYGEPTLRRAVPLALALISVS
NPRLNILDTLSKFSHDADPEVSYNSIFAMGMVGSGTNNARLAAMLRQLAQ
YHAKDPNNLFMVRLAQGLTHLGKGTLTLCPYHSDRQLMSQVAVAGLLTVL
VSFLDVRNIILGKSHYVLYGLVAAMQPRMLVTFDEELRPLPVSVRVGQAV
DVVGQAGKPKTITGFQTHTTPVLLAHGERAELATEEFLPVTPILEGFVIL
RKNPNYDL
[PSMD4]
SEQ ID NO: 2
MVLESTMVCVDNSEYMRNGDFLPTRLQAQQDAVNIVCHSKTRSNPENNVG
LITLANDCEVLTTLTPDTGRILSKLHTVQPKGKITFCTGIRVAHLALKHR
QGKNHKMRIIAFVGSPVEDNEKDLVKLAKRLKKEKVNVDIINFGEEEVNT
EKLTAFVNTLNGKDGTGSHLVTVPPGPSLADALISSPILAGEGGAMLGLG
ASDFEFGVDPSADPELALALRVSMEEQRQRQEEEARRAAAASAAEAGIAT
TGTEDSDDALLKMTISQQEFGRTGLPDLSSMTEEEQIAYAMQMSLQGAEF
GQAESADIDASSAMDTSEPAKEEDDYDVMQDPEFLQSVLENLPGVDPNNE
AIRNAMGSLASQATKDGKKDKKEEDKKRP
[ADRM1]
SEQ ID NO: 3
MTTSGALFPSLVPGSRGASNKYLVEFRAGKMSLKGTTVTPDKRKGLVYIQ
QTDDSLIHFCWKDRTSGNVEDDLIIFPDDCEFKRVPQCPSGRVYVLKFKA
GSKRLFFWMQEPKTDQDEEHCRKVNEYLNNPPMPGALGASGSSGHELSAL
GGEGGLQSLLGNMSHSQLMQLIGPAGLGGLGGLGALTGPGLASLLGSSGP
PGSSSSSSSRSQSAAVTPSSTTSSTRATPAPSAPAAASATSPSPAPSSGN
GASTAASPTQPIQLSDLQSILATMNVPAGPAGGQQVDLASVLTPEIMAPI
LANADVQERLLPYLPSGESLPQTADEIQNTLTSPQFQQALGMESAALASG
QLGPLMCQFGLPAEAVEAANKGDVEAFAKAMQNNAKPEQKEGDTKDKKDE
EEDMSLD

The target is a disease-causing target protein, and the target protein may be delivered directly to the proteasome by the fusion protein according to the present invention. The target-targeting moiety is a moiety capable of targeting the disease-causing target protein.

The term “moiety” refers to a functional group of a molecule, and means any substance that may specifically bind to a target protein. The target targeting moiety may include any substance that has binding affinity for the target. The target-targeting moiety may comprise any substance that has binding affinity for the target.

The target targeting moiety may be, for example, a nucleic acid molecule (DNA or RNA), a protein, an antibody, an antigen, an aptamer (RNA, DNA or peptide aptamer), or the like, without being limited thereto.

In a specific embodiment according to the present invention, the target-targeting moiety may be, for example, an antibody.

The term “antibody” includes not only a complete antibody form that specifically binds to the target, but also an antigen-binding fragment of the antibody molecule.

The term “complete antibody” refers to a structure having two full-length light chains and two full-length heavy chains, wherein each light-chain is linked to a corresponding heavy-chain by a disulfide bond.

As used herein, the term “heavy chain” is meant to encompass both a full-length heavy chain comprising a variable domain (VH), which comprises an amino acid sequence having a variable region sequence sufficient for imparting specificity to an antigen, and three constant domains (CH1, CH2 and CH3), and a fragment thereof. As used herein, the term “light chain” is meant to encompass both a full-length light chain comprising a variable domain (VL), which comprises an amino acid sequence having a variable region sequence sufficient for imparting specificity to an antigen, and a constant domain (CL), and a fragment thereof.

The whole antibody includes subtypes of IgA, IgD, IgE, IgM and IgG, and in particular, IgG includes IgG1, IgG2, IgG3 and IgG4. The heavy-chain constant region has gamma (γ), mu (p), alpha (α), delta (δ) and epsilon (ε) types, and is subclassified into gamma 1 (γ1), gamma 2 (γ2), gamma 3 (γ3), gamma 4 (γ4), alpha 1 (α1), and alpha 2 (α2). The light-chain constant region has kappa (κ) and lambda (λ) types.

The “antigen-binding fragment of an antibody” or “antibody fragment” refers to a fragment that has antigen-binding function and includes Fab, F(ab′), F(ab′)2, Fv and the like. Among the antibody fragments, Fab refers to a structure including a variable region of each of the heavy-chain and the light-chain, the constant region of the light-chain, and the first constant domain (CH1) of the heavy-chain, each having one antigen-binding site. Fab′ is different from Fab in that it has a hinge region including at least one cysteine residue at the C-terminus of the CH1 domain of the heavy-chain. F(ab′)2 is created by a disulfide bond between cysteine residues in the hinge region of Fab′.

Fv is the minimal antibody fragment having only a heavy-chain variable region and a light-chain variable region. Two-chain Fv is a fragment in which the variable region of the heavy-chain and the variable region of the light-chain are linked by a non-covalent bond, and single-chain Fv (scFv) is a fragment in which the variable region of the heavy-chain and the variable region of the light-chain are generally linked by a covalent bond via a peptide linker therebetween, or are directly linked at the C-terminal, forming a dimer-shaped structure, like the two-chain Fv. Such antibody fragments may be obtained using proteases (e.g., Fab can be obtained by restriction-cleaving the complete antibody with papain, and the F(ab′)2 fragment can be obtained by restriction-cleaving the complete antibody with pepsin), and may be produced using genetic recombination techniques.

The “Fv” fragment is an antibody fragment comprising complete antibody recognition and binding sites. Such a region is a dimer that consists of one heavy-chain variable domain and one light-chain variable domain linked to each other.

A “Fab” fragment comprises the variable and constant domains of the light chain, and the variable domain and first constant domain (CH1) of the heavy chain. A F(ab′)2 antibody fragment generally comprises a pair of Fab fragments covalently linked via a hinge cysteine located at the carboxyl terminus thereof.

The “single chain Fv (scFv) antibody fragment is a structure consisting of a single polypeptide chain comprising VH and VL domains of the antibody. The scFv antibody fragment may further comprise a polypeptide linker between the VH domain and the VL domain in order for the scFv to form a desired structure for antigen binding.

In one embodiment, the antibody or antigen-binding fragment according to the present invention may comprise monoclonal antibodies, multispecific antibodies, human antibodies, humanized antibodies, chimeric antibodies, scFVs, Fab fragments, F(ab′) fragments, disulfide-bond Fvs (sdFVs), anti-idiotypic (anti-Id) antibodies, or epitope-binding fragments of such antibodies.

The antibody may comprise single-domain antibodies, Fab, Fab′, scFv, diabodies, nanobodies, minibodies, tetrabodies, triabodies, or the like.

The heavy-chain constant region may be selected from gamma (γ), mu (u), alpha (α), delta (δ) and epsilon (c) isotypes. For example, the constant region may be gamma 1 (IgG1), gamma 3 (IgG3), or gamma 4 (IgG4). The light-chain constant region may be kappa or lambda.

The term “monoclonal antibody” refers to an identical antibody, which is obtained from a population of substantially homogeneous antibodies, that is, each antibody constituting the population, excluding possible naturally occurring mutations that may be present in trivial amounts. Monoclonal antibodies are highly specific and are thus induced against a single antigenic site. Unlike conventional (polyclonal) antibody preparations that typically include different antibodies directed against different determinants (epitopes), each monoclonal antibody is directed against a single determinant on the antigen.

The non-human (e.g., murine) antibody of the “humanized” form is a chimeric antibody containing a minimal sequence derived from non-human immunoglobulin. In most cases, the humanized antibody is a human immunoglobulin (receptor antibody) in which a residue from the hypervariable region of a receptor is replaced with a residue from the hypervariable region of a non-human species (donor antibody) such as a mouse, rat, rabbit or non-human primate having the desired specificity, affinity and ability.

The term “human antibody” means a molecule derived from human immunoglobulin, wherein the entire amino acid sequence constituting the antibody including a complementarity-determining region and a structural region are composed of human immunoglobulin.

A part of the heavy-chain and/or light-chain is identical to or homologous with the corresponding sequence in an antibody derived from a particular species or belonging to a particular antibody class or subclass, while the other chain(s) include “chimeric” antibodies (immunoglobulins) which are identical to or homologous with corresponding sequences in an antibody derived from another species or belonging to another antibody class or subclass, as well as fragments of such antibody exhibiting the desired biological activity.

As used herein, the term “antibody variable region” refers to the light- and heavy-chain regions of an antibody molecule comprising the amino acid sequences of a complementarity-determining region (CDR; i.e., CDR1, CDR2, and CDR3) and a framework region (FR). VH refers to a variable domain of the heavy chain. VL refers to a variable domain of the light chain.

The term “complementarity-determining region” (CDR) refers to an amino acid residue of the antibody variable domain, which is necessary for antigen binding. Each variable domain typically has three CDR regions, identified as CDR1, CDR2, and CDR3.

The term “framework region (FR)” refers to a variable domain residue other than a CDR residue. Each variable domain typically has four FRs, identified as FR1, FR2, FR3, and FR4.

The antibody or antigen-binding fragment thereof may be, for example, a single-chain variable fragment (scFv) or a nanobody.

The nanobody is an antibody fragment consisting of a single variable region fragment of an antibody and may be used interchangeably with “sdAb (single domain antibody)”. The nanobody is generally as defined in WO 2008/020079 or WO 2009/138519, and may include, but is not limited to, a VHH, a humanized VHH or a camelized VH (such as a camelized human VH).

The scFv is an antibody fragment, a structure consisting of a single polypeptide chain comprising the VH and VL domains of the antibody. The scFv may further comprise a polypeptide linker between the VH domain and the VL domain in order for the scFv to form a desired structure for antigen binding.

The linker may be a peptide linker and may have a length of about 10 to 25 aa. For example, it may comprise hydrophilic amino acids such as glycine and/or serine, without being limited thereto.

Specifically, the linker may comprise, for example, (GS)ₙ, (GGS)ₙ, (GSGGS)ₙ or (GₙS)ₘ (wherein n and m are each an integer from 1 to 10), but the linker may be, for example, (GₙS)ₘ (wherein n and m are each an integer from 1 to 10).

The antibody or antibody fragment may include not only any antibody but also biological equivalents thereto, as long as it can specifically recognize the target. For example, additional variations can be made to the amino acid sequence of the antibody in order to further improve the binding affinity and/or other biological properties of the antibody. Such variations include, for example, deletion, insertion and/or substitution of the amino acid sequence residues of the antibody. Such amino acid variations are based on the relative similarity of amino-acid side-chain substituents, such as the hydrophobicity, hydrophilicity, charge and size thereof. It can be seen through analysis of the size, shape and type of amino-acid side-chain substituents that all of arginine, lysine and histidine are positively charged residues; alanine, glycine and serine have similar sizes; and phenylalanine, tryptophan and tyrosine have similar shapes. Thus, based on these considerations, arginine, lysine and histidine; alanine, glycine and serine; and phenylalanine, tryptophan and tyrosine are considered to be biologically functional equivalents.

The fusion protein is prepared by combining two or more different proteins or homogeneous proteins, and has a structure in which a ubiquitin receptor and a target-targeting moiety are conjugated to each other.

The target-targeting moiety may be conjugated to the N- or C-terminus of the ubiquitin receptor.

The target-targeting moiety may be conjugated directly or via a linker to the ubiquitin receptor.

The linker may comprise a rigid linker or a flexible linker. The linker may be a peptide linker. The peptide linker may comprise at least one of a rigid linker and a flexible linker.

The rigid linker may have an amino acid sequence of A(EMAK)ₙA, wherein n may be an integer ranging from 1 to 5. The rigid linker may have an amino acid sequence of PAPAP or (XP)ₙ, wherein X may be alanine (Ala), lysine (Lys), or glutamic acid (Glu), and n may be an integer ranging from 5 to 17. The flexible linker may have an amino acid sequence of (G)ₙ, wherein n may be an integer ranging from 6 to 8. The linker may comprise, for example, (GS)ₙ, (GGS)ₙ, (GSGGS)ₙ or (GₙS)ₘ (wherein n and m are each an integer from 1 to 10), but the linker may be, for example, (GₙS)ₘ (wherein n and m are each an integer from 1 to 10).

The terms “polypeptide”, “peptide” and “protein” are used interchangeably herein to refer to a polymer of amino acid residues, wherein the polymer may be conjugated to a moiety that does consist of amino acids in some embodiments. The terms apply to amino acid polymers in which at least one amino acid residue is an artificial chemical mimetic of a corresponding naturally occurring as well as to naturally occurring amino acid polymers and non-naturally occurring amino acid polymers. A “fusion protein” refers to a chimeric protein encoding two or more separate protein sequences that are recombinantly expressed as a single moiety.

The term “amino acid” refers to naturally occurring and synthetic amino acids, as well as amino acid analogs and amino acid mimetics that function in a manner similar to the naturally occurring amino acids. Naturally occurring amino acids include those encoded by the genetic code, as well as those amino acids that are later modified, e.g., hydroxyproline, γ-carboxyglutamate, and O-phosphoserine. Amino acid analogs include compounds that have the same basic chemical structure as a naturally occurring amino acid, i.e., a carbon that is bound to a hydrogen, a carboxyl group, an amino group, and an R group, e.g., homoserine, norleucine, methionine sulfoxide, and methionine methyl sulfonium. Such analogs have modified R groups (e.g., norleucine) or modified peptide backbones, but may retain the same basic chemical structure as a naturally occurring amino acid Amino acid mimetics include chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

The present invention relates to an isolated nucleic acid encoding the fusion protein.

An “isolated nucleic acid” refers to a nucleic acid molecule that has been separated from at least about 50% of proteins, lipids, carbohydrates or other materials with which it is naturally found when isolated from the source cell, or is operably linked to a polypeptide with which it is not linked in nature, or does not occur in nature as part of a larger sequence. Specifically, the isolated nucleic acid molecule is substantially free from any other contaminating nucleic acid molecules or other contaminants that are found in its natural environment that would interfere with its therapeutic, diagnostic, prophylactic or research use.

The terms “polynucleotide”, “nucleic acid”, “nucleic acid molecule”, “nucleic acid oligomer”, “oligonucleotide”, “nucleic acid sequence”, and “nucleic acid fragment” are used interchangeably and are intended to include, but are not limited to, a polymeric form of nucleotides covalently linked together that may have various lengths, either deoxyribonucleotides or ribonucleotides, or analogs, derivatives or modifications thereof.

Different polynucleotides may have different three-dimensional structures, and may perform various functions, known or unknown. Examples of polynucleotides include, but are not limited to, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer.

The polynucleotide may typically include four nucleotide bases: adenine (A), cytosine (C), guanine (G), and thymine (T) (uracil (U) for thymine (T) when the polynucleotide is RNA).

The term “sequence” is the alphabetical representation of a molecule. This alphabetical representation can be input into databases in a computer having a central processing unit and used for bioinformatics applications such as functional genomics and homology searching. Polynucleotides may optionally include one or more non-standard nucleotides, nucleotide analogs and/or modified nucleotides.

The term “nucleic acid” refers to deoxyribonucleotides or ribonucleotides and polymers thereof in either single-, double- or multiple-stranded form, or complements thereof. The term “polynucleotide” refers to a linear sequence of nucleotides.

The term “nucleotide” generally refers to a single unit of a polynucleotide. Nucleotides may be ribonucleotides, deoxyribonucleotides, or modified versions thereof. Examples of polynucleotides include single and double stranded DNA, single and double stranded RNA (including siRNA), and hybrid molecules having mixtures of single and double stranded DNA and RNA.

Nucleic acids may be linear or branched. For example, nucleic acids may be a linear chain of nucleotides or the nucleic acids may be branched such that the nucleic acids comprise one or more arms or branches of nucleotides.

A nucleic acid, including a nucleic acid having a phosphothioate backbone, can comprise one or more reactive moieties. The reactive moiety can comprise any group capable of reacting with another molecule, such as a nucleic acid or a polypeptide, through covalent, noncovalent, or other interactions.

Nucleic acids, including nucleic acids with a phosphothioate backbone, may include one or more reactive moieties. The term “reactive moiety” includes any group capable of reacting with another molecule, e.g., a nucleic acid or polypeptide through non-covalent covalent, or other interactions. By way of example, the nucleic acid may include an amino acid reactive moiety that reacts with an amino acid on a protein or polypeptide through a covalent, non-covalent or other interaction.

“Conservatively modified variants” may apply to nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence which encodes a polypeptide also describes every possible silent variation of the nucleic acid. One skilled in the art can recognize that each codon in a nucleic acid (except AUG, which is ordinarily the only codon for methionine, and TGG, which is ordinarily the only codon for tryptophan) can be modified to yield a functionally identical molecule.

The term “isolated”, when applied to a nucleic acid or protein, denotes that the nucleic acid or protein is essentially free of other cellular components with which it is associated in the natural state. Purity and homogeneity may typically be determined using analytical chemistry techniques such as polyacrylamide gel electrophoresis or high-performance liquid chromatography.

“Complementarity” or “complementary” refers to the ability of a nucleic acid to form hydrogen bond(s) with another nucleic acid sequence by either traditional Watson-Crick pairing or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of nucleotides, or refers to two nucleic acids that hybridize under stringent conditions.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” includes a protein expressed from a particular gene.

The level of expression of a DNA molecule in a cell may be determined on the basis of either the amount of corresponding mRNA that is present within the cell or the amount of protein encoded by that DNA produced by the cell.

The present invention also relates to an expression vector comprising the nucleic acid.

The term “vector” refers to means for expressing a target gene in a host cell, and includes viral vectors such as plasmid vectors, cosmid vectors, bacteriophage vectors, adenovirus vectors, retrovirus vectors, and adeno-associated virus vectors. Components of a vector typically include, but are not limited to, one or more of the following: a signal sequence, an origin of replication, one or more antibiotic resistance marker genes, an enhancer element, a promoter, and a transcription termination sequence. A nucleic acid encoding an antibody is operably linked to the promoter and transcription termination sequence.

“Operably linked” means a functional linkage between a nucleic acid expression regulatory sequence (e.g., a promoter, a signal sequence, or an array of transcription factor binding sites) and another nucleic acid sequence, and thus the regulatory sequence regulates transcription and/or translation of the other nucleic acid sequence.

When a prokaryotic cell is used as a host, the vector generally contains a strong promoter capable of initiating transcription (e.g., tac promoter, lac promoter, lacUV5 promoter, lpp promoter, pLΔ promoter, pRΔ promoter, rac5 promoter, amp promoter, recA promoter, SP6 promoter, trp promoter, or T7 promoter), a ribosome binding site for initiating translation, and a transcription/translation a termination sequence. In addition, for example, when eukaryotic cell is used as a host, the vector may contain a promoter derived from the genome of mammalian cells (e.g., a metallothionein promoter, a β-actin promoter, a human hemoglobin promoter and a human muscle creatine promoter), or a promoter derived from mammalian virus (e.g., adenovirus late promoter, vaccinia virus 7.5K promoter, SV40 promoter, cytomegalovirus (CMV) promoter, HSV tk promoter, mouse breast tumor virus (MMTV) promoter, HIV LTR promoter, Moloney virus promoter, Epstein Barr virus (EBV) promoter, and Rous sarcoma virus (RSV) promoter), and generally has a polyadenylation sequence as a transcription termination sequence.

In some cases, the vector may be fused with another sequence to facilitate purification of the target protein. Examples of the sequence to be fused include glutathione S-transferase (Pharmacia, USA), maltose-binding protein (NEB, USA), FLAG (IBI, USA), and 6× His (hexahistidine; Quiagen, USA).

The vector contains an antibiotic resistance gene commonly used in the art as a selection marker, and examples of the antibiotic resistance gene include genes resistant to ampicillin, gentamycin, carbenicillin, chloramphenicol, streptomycin, kanamycin, geneticin, neomycin and tetracycline.

The present invention further relates to an isolated host cell comprising the expression vector.

Examples of host cells that may be used in the present invention include prokaryotic host cells, including Escherichia coli, Bacillus sp. strains such as Bacillus subtilis and Bacillus thuringiensis, Streptomyces, Pseudomonas (e.g., Pseudomonas putida), Proteus mirabilis, and Staphylococcus (e.g., Staphylococcus carnosus).

Examples of animal host cells that may be used in the present invention include, but are not limited to, COS-7, BHK, CHO, CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TRI, MRC 5, FS4, 3T3, RIN, A549, PC12, K562, PER.C6, SP2/0, NS-0, U20S, and HT1080.

The present invention also relates to a method for producing a fusion protein, comprising steps of: culturing the host cell to produce the fusion protein; and isolating and purifying the produced fusion protein.

The host cell may be cultured in various media. Any commercially available medium may be used as a culture medium without limitation. Any other necessary supplements known to those skilled in the art may also be included at appropriate concentrations. Culture conditions, such as temperature, pH, etc., are already used with the host cell selected for expression and will be apparent to those skilled in the art. Culture conditions, such as temperature, pH, etc., are those previously used with the host cell selected for expression and are known to those skilled in the art.

The fusion protein may be recovered by removing impurities by, for example, centrifugation or ultrafiltration, and purifying the resulting product using, for example, affinity chromatography or the like. Additional other purification techniques may be used, such as anion or cation exchange chromatography, hydrophobic interaction chromatography, hydroxyapatite chromatography, and the like.

The present invention also relates to a composition for degrading a target protein, comprising the fusion protein.

The target protein may be any disease-related protein related to a disease to be degraded. The target protein may be an exogenous or endogenous protein. The target protein may be an exogenous or endogenous protein that causes a disease. The present invention is a technology that may be applied universally by targeting and degrading various pathogenic proteins.

According to the present invention, not only was the effect of e-proteasome confirmed by targeting a target protein overexpressed by transfection in a cell, but its degradation effect on endogenous proteins was also confirmed.

In addition, transfection does not fully reflect the ability of ProMeD to degrade endogenous c-Fos. The present inventors confirmed that transduction may be used to overcome this experimental limitation.

The composition may be a pharmaceutical composition for preventing or treating a disease, comprising a pharmaceutically acceptable carrier.

The term “prevention” means any action of suppressing or delaying the progression of a disease by administering the composition according to the present invention, and the “treatment” means suppression, alleviation or elimination of the development of a disease.

An “effective amount” is generally an amount sufficient to reduce the severity and/or frequency of symptoms, eliminate the symptoms and/or their underlying cause, prevent the occurrence of symptoms and/or their underlying cause, and/or improve or remediate the damage that results from or is associated with a disease state. In some embodiments, the effective amount is a therapeutically effective amount or a prophylactically effective amount. A “therapeutically effective amount” is an amount sufficient to remedy a disease state or symptoms, particularly a state or symptoms associated with the disease state, or otherwise prevent, hinder, retard or reverse the progression of the disease state or any other undesirable symptom associated with the disease in any way whatsoever. A “prophylactically effective amount” is an amount of a pharmaceutical composition that, when administered to a subject, will have the intended prophylactic effect, e.g., preventing or delaying the onset (or reoccurrence) of a disease state, or reducing the likelihood of the onset (or reoccurrence) of a disease state.

The pharmaceutically acceptable carrier that is contained in the composition of the present invention is one commonly used for formulation, and examples thereof include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, water, syrup, methyl cellulose, methyl hydroxybenzoate, propyl hydroxybenzoate, talc, magnesium stearate, and mineral oil. In addition to these components, the composition of the present invention may further contain a lubricant, a wetting agent, a sweetener, a flavoring agent, an emulsifier, a suspending agent, a preservative, and the like.

The pharmaceutical composition of the present invention may be administered orally or parenterally. Where the pharmaceutical composition is to be administered parenterally, such as by intravenous, subcutaneous, ophthalmic, intraperitoneal, intramuscular, oral, rectal, intraorbital, intracerebral, intracranial, intraspinal, intraventricular, intrathecal, intracisternal, intracapsular, intranasal or aerosol administration, the pharmaceutical composition may comprise, for example, part of an aqueous or physiologically compatible fluid suspension or solution. Thus, the carrier or vehicle is physiologically acceptable, and thus may be added to the composition for delivery to a patient. Thus, the composition may generally include physiological saline as a carrier such as a fluid medium for formulation.

When administered orally, proteins or peptides are digested, and thus oral compositions should be formulated to coat the active agent or protect the same from degradation in the stomach. In addition, the pharmaceutical composition may be administered by any device that can deliver the active agent to a target cell.

The frequency of dosing will vary depending on pharmacokinetic parameters. Typically, the clinician will administer the pharmaceutical composition until a dosage is reached that achieves the desired result. Accordingly, the pharmaceutical composition may be administered as a single dose, as two or more doses over time, or as a continuous infusion via an implantation device or catheter. Further refinement of the appropriate dosage is routinely performed by those of ordinary skill in the art and is within the ambit of routinely performed tasks.

The appropriate dose of the composition according to the present invention varies depending on factors such as the formulation method, administration mode, patient's age, weight, sex, pathological diet, condition, administration time, administration route, excretion rate, and response sensitivity, and an ordinarily skilled physician can easily determine and prescribe the dose that is effective for the desired treatment or prevention. For example, the daily dose of the pharmaceutical composition of the present invention may be 0.0001 to 100 mg/kg.

Hereinafter, the present invention will be described in more detail by way of examples. These examples are only intended to illustrate the present invention, and it will be apparent to those skilled in the art that the scope of the present invention is not to be construed as being limited by these examples.

### Example 1: Construction of ProMED Platform

The present inventors sought to demonstrate the concept of a targeted protein degradation technology (ProMeD) platform that directly utilizes the proteasome, a final organelle that degrades proteins, as degradation machinery for pathogenic proteins. The present inventors conjugated a targeting antibody to a ubiquitin recognition subunit of a proteasome regulatory particle, thereby inducing an engineered proteasome (e-proteasome) that delivers a target protein directly to the proteasome. The present inventors checked the target protein degradation capacity of the induced e-proteasome and verified the specific pathway and ubiquitin dependency of the degradation.

The present inventors constructed a structure for expression of a protein by conjugating a targeting antibody to a ubiquitin recognition subunit (PSMD4) of a proteasome regulatory particle in order to induce an engineered proteasome (e-proteasome), and analyze the expression of the structure in mammalian cells.

There are three ubiquitin-recognition subunits (PSMD2, PSMD4, and ADRM1) in the proteasome regulatory particle. The present inventors hypothesized that, when an antibody is conjugated to the PSMD4 subunit, a target protein would be able to bind to the induced e-proteasome without ubiquitin tagging and be rapidly degraded.

A recombinant plasmid with scFv conjugated to the proteasome subunit PSMD4 was constructed through gene cloning.

The scFv is scFv(c-Fos), scFv(HA), scFv(Flag), or VHH(GFP) of SEQ ID NO: 15, 16, 17, or 18.

(SEQ ID NO: 15)
EVQLQQSGAELVRSGASVKLSCTASGFNIKDYYMHWVKQRPEQGLEWIGW
MDPKNGDTEYAPKFQGKATMTADTSSNTAYLQLSSLTSDDTAVYYCHDAI
TTPTRNFDVWGAGTTVTVSSGGGGSGGGGSGGGGSGGGSDVVMTQTPLSL
PVSLGDQASISCRSSQSLVHSNGNTYLHWYLQKPGQSPKLLMYKVSNRFS
GVPDRFSGSGSGTDFTLKISRVEAEDLGVYFCSQSTHVPFTFGSGTKLEI
K
(SEQ ID NO: 16)
EVKLVESGGGLVKPGGSLKLSCAASGFTFSSYGMSWVRQTPEKRLEWVAT
ISRGGSYTYYPDSVKGRFTISRDNAKNTLYLQMSSLRSEDTAIYYCARRE
TYDEKGFAYWGQGTTLTVSSGGGGSGGGGSGGGGSDIVLTQSPASLTVSL
GQRATISCKSSQSLLNSGNQKNYLTWYQQKPGQPPKLLIYWASTRESGIP
ARFSGSGSGTDFTLNIHPVEEEDAATYYCQNDNSHPLTFGAGTKLEIK
(SEQ ID NO: 17)
EVKLVESGGGLVKPGGSLKLSCAASGFTFSSFGMHWVRQTPEKRLEWVAY
ISSGSSTIYYADTVKGRFTISRDNAKNTLYLQMSSLRSEDTAIYYCARSL
ATAAFAYWGQGTTLTVSSGGGGSGGGGSGGGGSDIVLTQSPASLTVSLGQ
RATISCRSSQSIVYSNGNTYLEWYQQKPGQPPKLLIYKVSNRFSGIPARF
SGSGSGTDFTLNIHPVEEEDAATYYCFQGSHVPYTFGAGTKLEIKGAP
(SEQ ID NO: 18)
VQLVESGGALVQPGGSLRLSCAASGFPVNRYSMRWYRQAPGKEREWVAGM
SSAGDRSSYEDSVKGRFTISRDDARNTVYLQMNSLKPEDTAVYYCNVNVG
FEYWGQGTQVTVSS.

The sequence of PSMD4-scFv(c-Fos)-HTB (744 a.a.) is as follows:

(SEQ ID NO: 4)
MVLESTMVCVDNSEYMRNGDFLPTRLQAQQDAVNIVCHSKTRSNPENNVG
LITLANDCEVLTTLTPDTGRILSKLHTVQPKGKITFCTGIRVAHLALKHR
QGKNHKMRIIAFVGSPVEDNEKDLVKLAKRLKKEKVNVDIINFGEEEVNT
EKLTAFVNTLNGKDGTGSHLVTVPPGPSLADALISSPILAGEGGAMLGLG
ASDFEFGVDPSADPELALALRVSMEEQRQRQEEEARRAAAASAAEAGIAT
TGTEDSDDALLKMTISQQEFGRTGLPDLSSMTEEEQIAYAMQMSLQGAEF
GQAESADIDASSAMDTSEPAKEEDDYDVMQDPEFLQSVLENLPGVDPNNE
AIRNAMGSLASQATKDGKKDKKEEDKKRPKLGTELGSMEVQLQQSGAELV
RSGASVKLSCTASGFNIKDYYMHWVKQRPEQGLEWIGWMDPKNGDTEYAP
KFQGKATMTADTSSNTAYLQLSSLTSDDTAVYYCHDAITTPTRNFDVWGA
GTTVTVSSGGGGSGGGGSGGGGSGGGSDVVMTQTPLSLPVSLGDQASISC
RSSQSLVHSNGNTYLHWYLQKPGQSPKLLMYKVSNRFSGVPDRFSGSGSG
TDFTLKISRVEAEDLGVYFCSQSTHVPFTFGSGTKLEIKHHHHHHDYDIP
TTASENLYFQGELGMRGSAGKAGEGEIPAPLAGTVSKILVKEGDTVKAGQ
TVLVLEAMKMETEINAPTDGKVEKVLVKERNAVQGGQGLIKIGV.

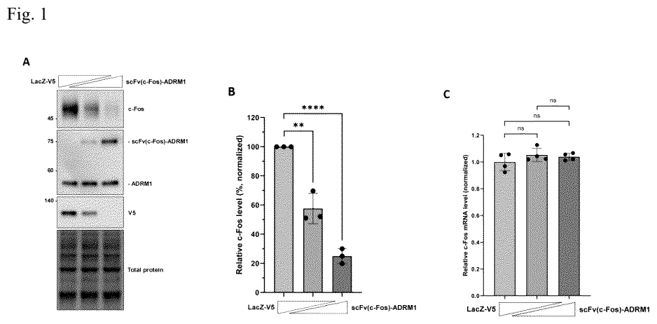

A linker may be included. It may comprise a GS: GGGGS linker or an EA: EAAAK linker, the sequences of which are as follows. The sequence of PSMD4-scFv was selected because recognition of ubiquitin chains occurs at the C-terminal region of PSMD4. In addition, a recombinant plasmid having a linker between the subunit and the antibody was also constructed and it was checked whether there was a difference in the target protein degradation ability (FIG. 1). FIG. 1 in A therein is a schematic diagram showing recombinant fusion protein plasmids having different types of linkers (no linker, flexible linker ((GGGGS) 3), and rigid linker ((EAAAK) 3).

The sequence of PSMD4-GS-scFv(c-Fos)-HTB (759 a.a.) is as follows:

(SEQ ID NO: 5)
MVLESTMVCVDNSEYMRNGDFLPTRLQAQQDAVNIVCHSKTRSNPENNVG
LITLANDCEVLTTLTPDTGRILSKLHTVQPKGKITFCTGIRVAHLALKHR
QGKNHKMRIIAFVGSPVEDNEKDLVKLAKRLKKEKVNVDIINFGEEEVNT
EKLTAFVNTLNGKDGTGSHLVTVPPGPSLADALISSPILAGEGGAMLGLG
ASDFEFGVDPSADPELALALRVSMEEQRQRQEEEARRAAAASAAEAGIAT
TGTEDSDDALLKMTISQQEFGRTGLPDLSSMTEEEQIAYAMQMSLQGAEF
GQAESADIDASSAMDTSEPAKEEDDYDVMQDPEFLQSVLENLPGVDPNNE
AIRNAMGSLASQATKDGKKDKKEEDKKRPKLGTELGSMGGGGSGGGGSGG
GGSEVQLQQSGAELVRSGASVKLSCTASGFNIKDYYMHWVKQRPEQGLEW
IGWMDPKNGDTEYAPKFQGKATMTADTSSNTAYLQLSSLTSDDTAVYYCH
DAITTPTRNFDVWGAGTTVTVSSGGGGSGGGGSGGGGSGGGSDVVMTQTP
LSLPVSLGDQASISCRSSQSLVHSNGNTYLHWYLQKPGQSPKLLMYKVSN
RFSGVPDRFSGSGSGTDFTLKISRVEAEDLGVYFCSQSTHVPFTFGSGTK
LEIKHHHHHHDYDIPTTASENLYFQGELGMRGSAGKAGEGEIPAPLAGTV
SKILVKEGDTVKAGQTVLVLEAMKMETEINAPTDGKVEKVLVKERNAVQG
GQGLIKIGV.

The sequence of PSMD4-EA-scFv(c-Fos)-HTB (759 a.a.) is as follows:

(SEQ ID NO: 6)
MVLESTMVCVDNSEYMRNGDFLPTRLQAQQDAVNIVCHSKTRSNPENNVG
LITLANDCEVLTTLTPDTGRILSKLHTVQPKGKITFCTGIRVAHLALKHR
QGKNHKMRIIAFVGSPVEDNEKDLVKLAKRLKKEKVNVDIINFGEEEVNT
EKLTAFVNTLNGKDGTGSHLVTVPPGPSLADALISSPILAGEGGAMLGLG
ASDFEFGVDPSADPELALALRVSMEEQRQRQEEEARRAAAASAAEAGIAT
TGTEDSDDALLKMTISQQEFGRTGLPDLSSMTEEEQIAYAMQMSLQGAEF
GQAESADIDASSAMDTSEPAKEEDDYDVMQDPEFLQSVLENLPGVDPNNE
AIRNAMGSLASQATKDGKKDKKEEDKKRPKLGTELGSMEAAAKEAAAKEA
AAKEVQLQQSGAELVRSGASVKLSCTASGFNIKDYYMHWVKQRPEQGLEW
IGWMDPKNGDTEYAPKFQGKATMTADTSSNTAYLQLSSLTSDDTAVYYCH
DAITTPTRNFDVWGAGTTVTVSSGGGGSGGGGSGGGGSGGGSDVVMTQTP
LSLPVSLGDQASISCRSSQSLVHSNGNTYLHWYLQKPGQSPKLLMYKVSN
RFSGVPDRFSGSGSGTDFTLKISRVEAEDLGVYFCSQSTHVPFTFGSGTK
LEIKHHHHHHDYDIPTTASENLYFQGELGMRGSAGKAGEGEIPAPLAGTV
SKILVKEGDTVKAGQTVLVLEAMKMETEINAPTDGKVEKVLVKERNAVQG
GQGLIKIGV.

Recombinant plasmids comprising PSMD2 ADRM1, respectively, can also be constructed to have the same structure as the recombinant plasmid comprising PSMD4.

ADRM1-scFv (c-Fos)-HTB (772 a.a.)
(SEQ ID NO: 7)
MTTSGALFPSLVPGSRGASNKYLVEFRAGKMSLKGTTVTPDKRKGLVYIQQTDDS
LIHFCWKDRTSGNVEDDLIIFPDDCEFKRVPQCPSGRVYVLKFKAGSKRLFFWMQEPKTDQ
DEEHCRKVNEYLNNPPMPGALGASGSSGHELSALGGEGGLQSLLGNMSHSQLMQLIGPAGL
GGLGGLGALTGPGLASLLGSSGPPGSSSSSSSRSQSAAVTPSSTTSSTRATPAPSAPAAAS
ATSPSPAPSSGNGASTAASPTQPIQLSDLQSILATMNVPAGPAGGQQVDLASVLTPEIMAP
ILANADVQERLLPYLPSGESLPQTADEIQNTLTSPQFQQALGMFSAALASGQLGPLMCQFG
LPAEAVEAANKGDVEAFAKAMQNNAKPEQKEGDTKDKKDEEEDMSLDKLGTELGSMEVQLQ
QSGAELVRSGASVKLSCTASGFNIKDYYMHWVKQRPEQGLEWIGWMDPKNGDTEYAPKFQG
KATMTADTSSNTAYLQLSSLTSDDTAVYYCHDAITTPTRNFDVWGAGTTVTVSSGGGGSGG
GGSGGGGSGGGSDVVMTQTPLSLPVSLGDQASISCRSSQSLVHSNGNTYLHWYLQKPGQSP
KLLMYKVSNRFSGVPDRFSGSGSGTDFTLKISRVEAEDLGVYFCSQSTHVPFTFGSGTKLE
IKHHHHHHDYDIPTTASENLYFQGELGMRGSAGKAGEGEIPAPLAGTVSKILVKEGDTVKA
GQTVLVLEAMKMETEINAPTDGKVEKVLVKERDAVQGGQGLIKIGV.
BTH-scFv (c-Fos)-ADRM1 (765 a.a.)
(SEQ ID NO: 8)
MAGKAGEGEIPAPFAGTVFKILVKEGDTVKAGQTVLVLEAMKMETEINAPTDGKV
EKVLVKERDAVQGGQGLIKIGDYDIPTTASENLYFQGELGMRGSHHHHHHEVQLQQSGAEL
VRSGASVKLSCTASGFNIKDYYMHWVKQRPEQGLEWIGWMDPKNGDTEYAPKFQGKATMTA
DTSSNTAYLQLSSLTSDDTAVYYCHDAITTPTRNFDVWGAGTTVTVSSGGGGSGGGGSGGG
GSGGGSDVVMTQTPLSLPVSLGDQASISCRSSQSLVHSNGNTYLHWYLQKPGQSPKLLMYK
VSNRFSGVPDRFSGSGSGTDFTLKISRVEAEDLGVYFCSQSTHVPFTFGSGTKLEIKEFMT
TSGALFPSLVPGSRGASNKYLVEFRAGKMSLKGTTVTPDKRKGLVYIQQTDDSLIHFCWKD
RTSGNVEDDLIIFPDDCEFKRVPQCPSGRVYVLKFKAGSKRLFFWMQEPKTDQDEEHCRKV
NEYLNNPPMPGALGASGSSGHELSALGGEGGLQSLLGNMSHSQLMQLIGPAGLGGLGGLGA
LTGPGLASLLGSSGPPGSSSSSSSRSQSAAVTPSSTTSSTRATPAPSAPAAASATSPSPAP
SSGNGASTAASPTQPIQLSDLQSILATMNVPAGPAGGQQVDLASVLTPEIMAPILANADVQ
ERLLPYLPSGESLPQTADEIQNTLTSPQFQQALGMFSAALASGQLGPLMCQFGLPAEAVEA
ANKGDVEAFAKAMQNNAKPEQKEGDTKDKKDEEEDMSLD.
BTH-scFv (c-Fos)-PSMD2 (1266 a.a.)
(SEQ ID NO: 9)
MAGKAGEGEIPAPFAGTVFKILVKEGDTVKAGQTVLVLEAMKMETEINAPTDGKV
EKVLVKERDAVQGGQGLIKIGDYDIPTTASENLYFQGELGMRGSHHHHHHEVQLQQSGAEL
VRSGASVKLSCTASGFNIKDYYMHWVKQRPEQGLEWIGWMDPKNGDTEYAPKFQGKATMTA
DTSSNTAYLQLSSLTSDDTAVYYCHDAITTPTRNFDVWGAGTTVTVSSGGGGGGGGSGGG
GSGGGSDVVMTQTPLSLPVSLGDQASISCRSSQSLVHSNGNTYLHWYLQKPGQSPKLLMYK
VSNRFSGVPDRFSGSGSGTDFTLKISRVEAEDLGVYFCSQSTHVPFTFGSGTKLEIKEFME
EGGRDKAPVQPQQSPAAAPGGTDEKPSGKERRDAGDKDKEQELSEEDKQLQDELEMLVERL
GEKDTSLYRPALEELRRQIRSSTTSMTSVPKPLKFLRPHYGKLKEIYENMAPGENKRFAAD
IISVLAMTMSGERECLKYRLVGSQEELASWGHEYVRHLAGEVAKEWQELDDAEKVQREPLL
TLVKEIVPYNMAHNAEHEACDLLMEIEQVDMLEKDIDENAYAKVCLYLTSCVNYVPEPENS
ALLRCALGVFRKFSRFPEALRLALMLNDMELVEDIFTSCKDVVVQKQMAFMLGRHGVFLEL
SEDVEEYEDLTEIMSNVQLNSNFLALARELDIMEPKVPDDIYKTHLENNRFGGSGSQVDSA
RMNLASSFVNGFVNAAFGQDKLLTDDGNKWLYKNKDHGMLSAAASLGMILLWDVDGGLTQI
DKYLYSSEDYIKSGALLACGIVNSGVRNECDPALALLSDYVLHNSNTMRLGSIFGLGLAYA
GSNREDVLTLLLPVMGDSKSSMEVAGVTALACGMIAVGSCNGDVTSTILQTIMEKSETELK
DTYARWLPLGLGLNHLGKGEAIEAILAALEVVSEPFRSFANTLVDVCAYAGSGNVLKVQQL
LHICSEHFDSKEKEEDKDKKEKKDKDKKEAPADMGAHQGVAVLGIALIAMGEEIGAEMALR
TFGHLLRYGEPTLRRAVPLALALISVSNPRLNILDTLSKFSHDADPEVSYNSIFAMGMVGS
GTNNARLAAMLRQLAQYHAKDPNNLFMVRLAQGLTHLGKGTLTLCPYHSDRQLMSQVAVAG
LLTVLVSFLDVRNIILGKSHYVLYGLVAAMQPRMLVTFDEELRPLPVSVRVGQAVDVVGQA
GKPKTITGFQTHTTPVLLAHGERAELATEEFLPVTPILEGFVILRKNPNYDL.
BTH-scFv (HA)-ADRM1 (770 a.a.)
(SEQ ID NO: 10)
MAGKAGEGEIPAPFAGTVFKILVKEGDTVKAGQTVLVLEAMKMETEINAPTDGKV
EKVLVKERDAVQGGQGLIKIGDYDIPTTASENLYFQGELGMRGSHHHHHHLINEVKLVESG
GGLVKPGGSLKLSCAASGFTFSSYGMSWVRQTPEKRLEWVATISRGGSYTYYPDSVKGRFT
ISRDNAKNTLYLQMSSLRSEDTAIYYCARRETYDEKGFAYWGQGTTLTVSSGGGGGGGGS
GGGGSDIVLTQSPASLTVSLGQRATISCKSSQSLLNSGNQKNYLTWYQQKPGQPPKLLIYW
ASTRESGIPARFSGSGSGTDFTLNIHPVEEEDAATYYCQNDNSHPLTFGAGTKLEIKRAAA
GAPMTTSGALFPSLVPGSRGASNKYLVEFRAGKMSLKGTTVTPDKRKGLVYIQQTDDSLIH
FCWKDRTSGNVEDDLIIFPDDCEFKRVPQCPSGRVYVLKFKAGSKRLFFWMQEPKTDQDEE
HCRKVNEYLNNPPMPGALGASGSSGHELSALGGEGGLQSLLGNMSHSQLMQLIGPAGLGGL
GGLGALTGPGLASLLGSSGPPGSSSSSSSRSQSAAVTPSSTTSSTRATPAPSAPAAASATS
PSPAPSSGNGASTAASPTQPIQLSDLQSILATMNVPAGPAGGQQVDLASVLTPEIMAPILA
NADVQERLLPYLPSGESLPQTADEIQNTLTSPQFQQALGMFSAALASGQLGPLMCQFGLPA
EAVEAANKGDVEAFAKAMQNNAKPEQKEGDTKDKKDEEEDMSLD.
PSMD4-scFv (Flag)-BTH (737 a.a.)
(SEQ ID NO: 11)
MVLESTMVCVDNSEYMRNGDFLPTRLQAQQDAVNIVCHSKTRSNPENNVGLITLA
NDCEVLTTLTPDTGRILSKLHTVQPKGKITFCTGIRVAHLALKHRQGKNHKMRIIAFVGSP
VEDNEKDLVKLAKRLKKEKVNVDIINFGEEEVNTEKLTAFVNTLNGKDGTGSHLVTVPPGP
SLADALISSPILAGEGGAMLGLGASDFEFGVDPSADPELALALRVSMEEQRQRQEEEARRA
AAASAAEAGIATTGTEDSDDALLKMTISQQEFGRTGLPDLSSMTEEEQIAYAMQMSLQGAE
FGQAESADIDASSAMDTSEPAKEEDDYDVMQDPEFLQSVLENLPGVDPNNEAIRNAMGSLA
SQATKDGKKDKKEEDKKRPLINMAEVKLVESGGGLVKPGGSLKLSCAASGFTFSSFGMHWV
RQTPEKRLEWVAYISSGSSTIYYADTVKGRFTISRDNAKNTLYLQMSSLRSEDTAIYYCAR
SLATAAFAYWGQGTTLTVSSGGGGSGGGGSGGGGSDIVLTQSPASLTVSLGQRATISCRSS
QSIVYSNGNTYLEWYQQKPGQPPKLLIYKVSNRFSGIPARFSGSGSGTDFTLNIHPVEEED
AATYYCFQGSHVPYTFGAGTKLEIKGAPHHHHHHDYDIPTTASENLYFQGELGMRGSAGKA
GEGEIPAPLAGTVSKILVKEGDTVKAGQTVLVLEAMKMETEINAPTDGKVEKVLVKERDAV
QGGQGLIKIGV.
BTH-VHH (GFP)-ADRM1 (632 a.a.)
(SEQ ID NO: 12)
MAGKAGEGEIPAPFAGTVFKILVKEGDTVKAGQTVLVLEAMKMETEINAPTDGKV
EKVLVKERDAVQGGQGLIKIGDYDIPTTASENLYFQGELGMRGSHHHHHHLINVQLVESGG
ALVQPGGSLRLSCAASGFPVNRYSMRWYRQAPGKEREWVAGMSSAGDRSSYEDSVKGRFTI
SRDDARNTVYLQMNSLKPEDTAVYYCNVNVGFEYWGQGTQVTVSSGAPMTTSGALFPSLVP
GSRGASNKYLVEFRAGKMSLKGTTVTPDKRKGLVYIQQTDDSLIHFCWKDRTSGNVEDDLI
IFPDDCEFKRVPQCPSGRVYVLKFKAGSKRLFFWMQEPKTDQDEEHCRKVNEYLNNPPMPG
ALGASGSSGHELSALGGEGGLQSLLGNMSHSQLMQLIGPAGLGGLGGLGALTGPGLASLLG
SSGPPGSSSSSSSRSQSAAVTPSSTTSSTRATPAPSAPAAASATSPSPAPSSGNGASTAAS
PTQPIQLSDLQSILATMNVPAGPAGGQQVDLASVLTPEIMAPILANADVQERLLPYLPSGE
SLPQTADEIQNTLTSPQFQQALGMFSAALASGQLGPLMCQFGLPAEAVEAANKGDVEAFAK
AMQNNAKPEQKEGDTKDKKDEEEDMSLD.
PSMD4-VHH (GFP)-BTH (604 a.a.)
(SEQ ID NO: 13)
MVLESTMVCVDNSEYMRNGDFLPTRLQAQQDAVNIVCHSKTRSNPENNVGLITLA
NDCEVLTTLTPDTGRILSKLHTVQPKGKITFCTGIRVAHLALKHRQGKNHKMRIIAFVGSP
VEDNEKDLVKLAKRLKKEKVNVDIINFGEEEVNTEKLTAFVNTLNGKDGTGSHLVTVPPGP
SLADALISSPILAGEGGAMLGLGASDFEFGVDPSADPELALALRVSMEEQRQRQEEEARRA
AAASAAEAGIATTGTEDSDDALLKMTISQQEFGRTGLPDLSSMTEEEQIAYAMQMSLQGAE
FGQAESADIDASSAMDTSEPAKEEDDYDVMQDPEFLQSVLENLPGVDPNNEAIRNAMGSLA
SQATKDGKKDKKEEDKKRPLINVQLVESGGALVQPGGSLRLSCAASGFPVNRYSMRWYRQA
PGKEREWVAGMSSAGDRSSYEDSVKGRFTISRDDARNTVYLQMNSLKPEDTAVYYCNVNVG
FEYWGQGTQVTVSSGAPHHHHHHDYDIPTTASENLYFQGELGMRGSAGKAGEGEIPAPLAG
TVSKILVKEGDTVKAGQTVLVLEAMKMETEINAPTDGKVEKVLVKERDAVQGGQGLIKIGV.

To check whether the constructed recombinant plasmids are stably expressed in mammalian cells, the expression level of each recombinant plasmid was analyzed by transfection. It was confirmed by immunoblotting that the recombinant fusion proteins were expressed in the A549 cell line. It was confirmed that, when the PSMD4-linker-scFv plasmids were overexpressed by transfection in mammalian cells (A549), they were expressed efficiently.

### Example 2: Target Protein Degradation by Recombinant Fusion Protein (scFv-ADRM1)

### 1. Evaluation of Protein Degradation Ability

It was observed that, when the recombinant fusion protein according to Example 1 was expressed in cells, the target protein was significantly reduced compared to the control, indicating that the recombinant fusion protein has excellent degradation ability.

In addition, it was confirmed that, when the recombinant protein was expressed in a dose-dependent manner, the target protein was gradually degraded, indicating that the degradation was caused by the recombinant protein (FIG. 1 in A and B therein).

To determine whether the transcription level of the target protein was the same in all groups, mRNA was extracted and synthesized into cDNA, and it was confirmed by qPCR that the mRNA level of the target protein c-Fos was the same in all groups (FIG. 1 in C therein).

This suggests that the decrease in the target protein (c-Fos) is not due to changes at the transcription level but is due to protein degradation.

### 2. Protein Degradation Ability According to Assembly Location

Not only the C-terminus but also the N-terminus of the ubiquitin recognition subunit ADRM1 is outside the proteasome, and thus the subunit ADRM1 can be assembled into the proteasome without any problem when an antibody is conjugated thereto. Under the hypothesis that target protein degradation would be possible even when an antibody was conjugated at the C-terminus, gene cloning was performed.

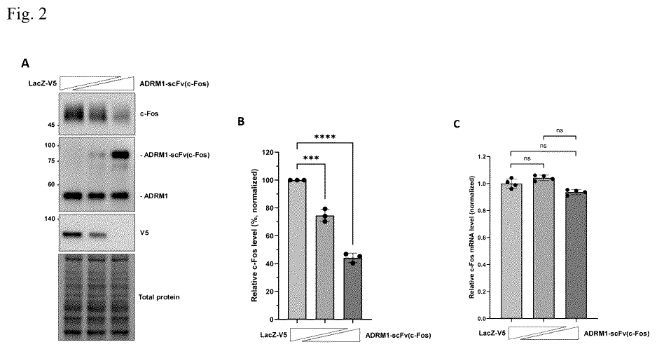

A recombinant fusion protein (ADRM1-scFv(c-Fos)) with an antibody attached to the C-terminus also exhibited good degradation ability (FIG. 2 in A and B therein). In addition, as a result of mRNA extraction and qPCR, it was confirmed that the mRNA levels of the target protein c-Fos were the same in all groups (FIG. 2 in C therein). This suggests that recombinant proteins that induce target protein degradation can be constructed using both termini of the ubiquitin recognition subunit ADRM1.

### 3. Checking of Whether Stability is Decreased

To examine whether the recombinant protein not only degrades the target protein but also reduces its stability, an experiment was conducted using cycloheximide, a translation inhibitor in cells. By tracking the quantitative changes in the target protein (c-Fos) over time after treatment with the translation inhibitor, the stability and half-life of c-Fos can be determined (chase experiment).

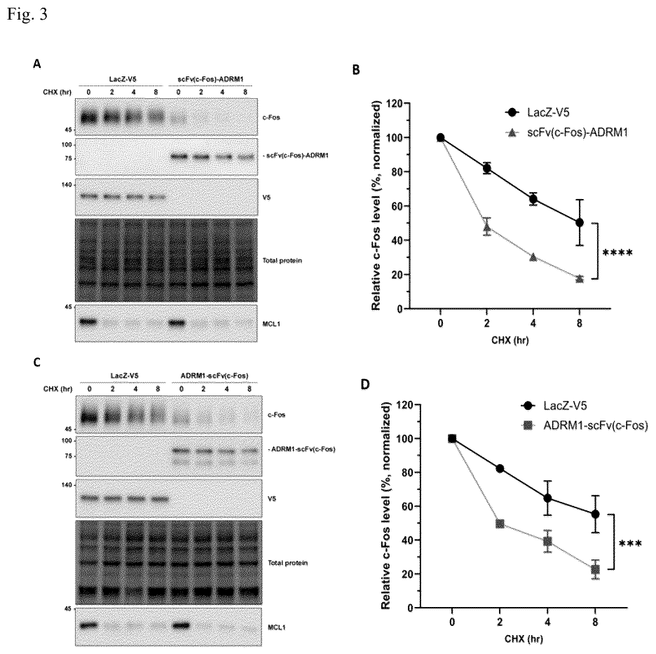

It could be confirmed that the target protein (c-Fos) was degraded much faster in the group in which each recombinant protein was overexpressed than in the control group. This suggests that the recombinant protein not only degrades the target protein but also reduces its stability, and these results are reliable in that the degradation ability and the degree of stability reduction are proportional (FIG. 3 in A to D therein).

4. Ability to Degrade Target Proteins Other than c-Fos

In order for ProMeD to be a platform technology, it should be able to target and degrade other proteins in addition to the target protein c-Fos. To confirm this ability, the present inventors utilized an antibody that binds to HA-tag (hemagglutinin tag) and an antibody that binds to GFP.

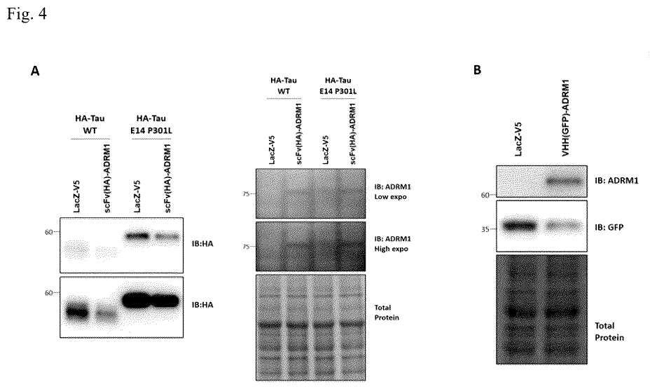

A recombinant protein in which another antibody that binds HA (scFv(HA)) is conjugated to the N-terminus of ADRM1 effectively degraded HA-tau compared to the control. This effect was observed not only in the wild-type HA-tau but also in mutant forms associated with the onset of dementia (FIG. 4 in A therein). In addition, the recombinant protein in which the antibody that binds GFP (VHH(GFP)) is conjugated to the N-terminus of ADRM1 degraded GFP-ODC well (FIG. 4 in B therein). GFP-ODC is a protein that is degraded by the proteasome without ubiquitination, and this result shows that ProMeD can promote ubiquitin-independent degradation.

### Example 3: Degradation of Target Protein by Recombinant Fusion Protein (PSMD4-scFv)

In addition to ADRM1, there are two other ubiquitin-recognition subunits in the proteasome. To analyze whether target protein degradation is possible even by using one of these subunits, PSMD4, the present inventors constructed a recombinant protein in which an antibody that binds to c-Fos (scFv(c-Fos)) is conjugated to PSMD4.

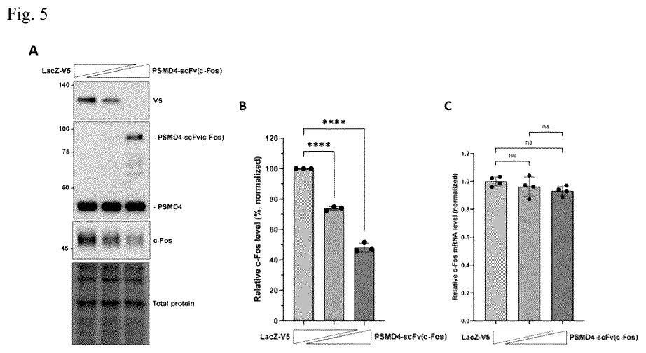

The recombinant protein (PSMD4-scFv(c-Fos)) also exhibited the ability to degrade the target protein (FIG. 5 in A therein). In addition, it was confirmed that the degradation occurred in a manner dependent on the dose of the recombinant protein, and it was confirmed by qPCR that the mRNA level of the target protein c-Fos was the same in all groups (FIG. 5 in B and C therein).

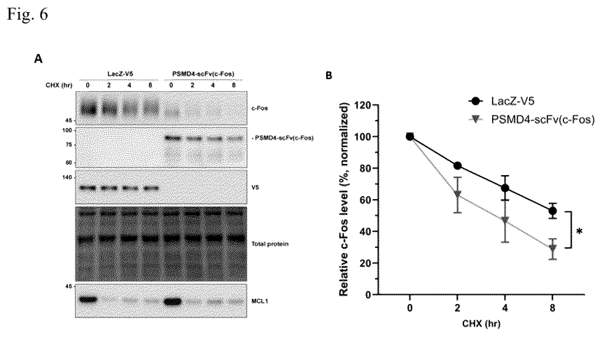

It was observed that the target protein (c-Fos) was degraded faster in the presence of the recombinant protein than in the control. Although the difference was smaller than that in the case of ProMeD utilizing the ADRM1 subunit, it was confirmed to be statistically significant (FIG. 6 in A and B therein).

The above results suggest that ProMeD is a platform technology that can induce target protein degradation even by using other ubiquitin recognition subunits, such as PSMD4, in addition to ADRM1. For degradation of c-Fos, it is ideal to have an antibody at the N-terminus of ADRM1, but this may vary depending on the substrate.

The recombinant protein of ProMeD is largely composed of a ubiquitin recognition subunit and an antibody. A degradation assay was performed to determine whether the observed degradation of the target protein was due to the recombinant protein or simply due to overexpression of the subunit or antibody.

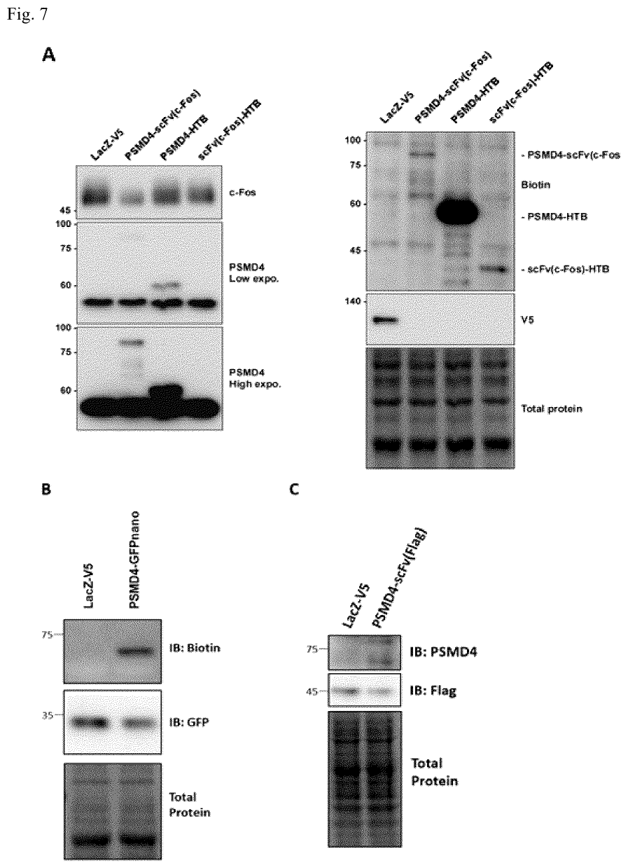

The levels of the target protein (c-Fos) in the group overexpressing the ubiquitin recognition subunit alone and the group overexpressing the antibody alone were similar to that in the control group (Lacz-V5), and clear degradation was observed only when the recombinant protein was overexpressed (FIG. 7 in A therein).

This indicates that the degradation of ProMeD is dependent on the recombinant protein, not on each component of the recombinant protein. The recombinant protein in which another antibody that binds to Flag is conjugated to PSMD4 exhibited degradation ability for Flag-TDP43, and even the recombinant protein in which an antibody that binds to GFP is conjugated to PSMD4 exhibited degradation ability for GFP-ODC (FIG. 7 in B and C therein).

This suggests that, like ADRM1, PSMD4 can target and degrade a variety of proteins, demonstrating the universal nature of ProMeD.

### Example 4: Mechanism of Target Protein Degradation by ProMeD

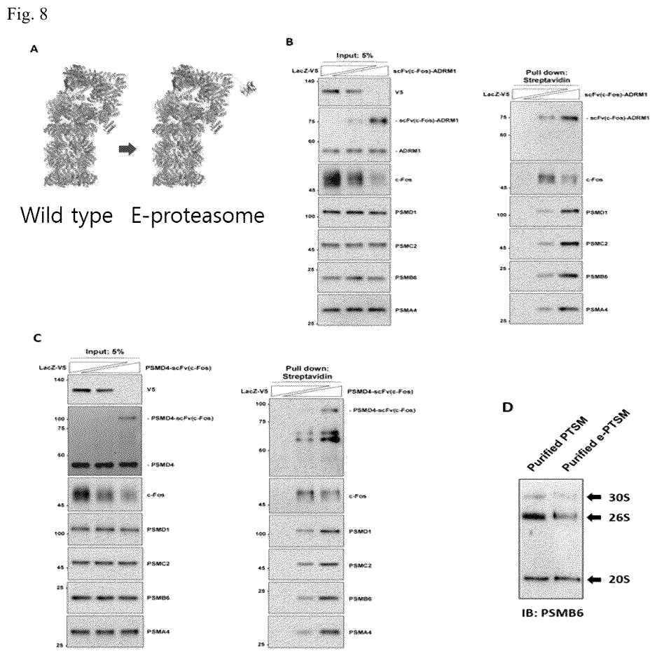

Engineered ADRM1 and PSMD4 have been shown to be capable of degrading target proteins. A key hypothesis for the mechanism of ProMeD is that the recombinant protein is assembled into the proteasome, and the resulting e-proteasome binds to the target substrate, resulting in degradation (FIG. 8 in A therein).

To prove this hypothesis, it is necessary to confirm the existence of e-proteasomes, formed by proteasome engineering, in a situation where degradation of the target protein occurs.

In a situation where the target protein gradually decreased with dose-dependent expression of the recombinant fusion protein (scFv(c-Fos)-ADRM1), the recombinant protein could be purified using the affinity between the biotin of the HTB tag and streptavidin beads. The purified protein contained subunits of both the central and regulatory particles of the proteasome, suggesting that the recombinant protein could assemble into the proteasome (FIG. 8 in B therein).

Additionally, it can be seen that the purified proteasome subunits increased as the dose of the recombinant protein increased, which suggests that more e-proteasomes are likely to be formed as the dose of the recombinant protein increases. The gradual increase in e-proteasomes with a gradual decrease in the target protein is a result that supports the core hypothesis, although it is only at the level of correlation. Even in the case of the protein using PSMD4 (PSMD4-scFv(c-Fos)), similar results were obtained, and c-Fos was pulled out only in the experimental group in which the recombinant protein was purified, indicating binding between the substrate and the antibody (FIG. 8 in C therein). As a result of analyzing the purified protein while preserving the structure by native-PAGE rather than SDS-PAGE, 30S, 26S, and 20S proteasomes could all be observed (FIG. 8 in D therein). This suggests that the recombinant protein assembles into the proteasome, leading to the induction of a fully formed e-proteasome.

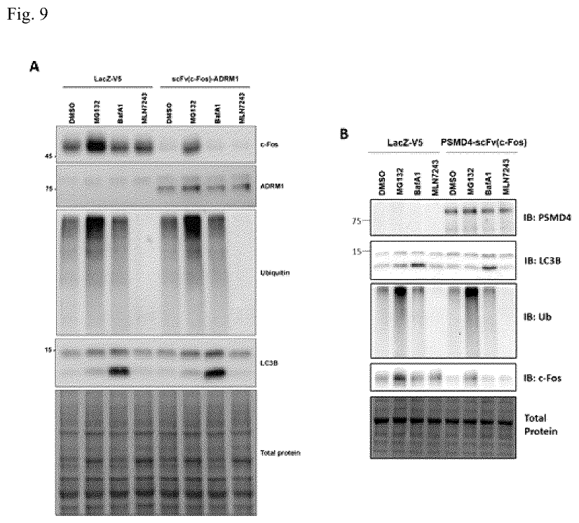

To determine whether the mechanism of ProMeD is dependent on the e-proteasome, the degradation pathway was analyzed using a proteasome inhibitor (MG132), a lysosomal degradation pathway inhibitor (BafA1), and an E1 enzyme inhibitor (MLN7243). In both recombinant proteins, it was shown that, only upon treatment with the proteasome inhibitor, the degradation of the target protein was inhibited and c-Fos was accumulated (FIG. 9 in A and B therein). This suggests that the ProMeD pathway is independent of lysosomal degradation pathways such as autophagy, and is dependent on the proteasome. The fact that the target protein is degraded in proportion to the dose of e-proteasome (FIG. 8 in B to C therein) and that the degradation ability disappears when the proteasome is inhibited clearly supports the core hypothesis of the mechanism. In addition, the fact that treatment with the E1 enzyme inhibitor (MLN7243) that inhibits ubiquitination has no effect on the degradation ability is evidence that ProMeD is a ubiquitin-independent system (FIG. 9 in A and B therein).

Recombinant protein-induced e-proteasomes were observed to interact with the target protein (FIG. 8 in B and C therein). This affinity-dependent degradation induced by binding of antibodies to substrates is one of the key factors in demonstrating the target specificity and mechanism of action of ProMeD. This degradation that is caused by the binding between the antibody and the substrates, that is, the affinity dependence, is one of the key factors in demonstrating the target specificity and mechanism of action of ProMeD.

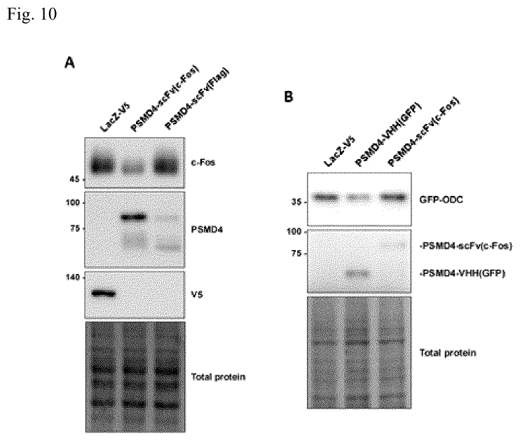

To demonstrate the affinity dependence of ProMeD, a recombinant protein that has no affinity for the target protein was used. The group overexpressing the recombinant protein (PSMD4-scFv(Flag)) that does not bind to the target protein (c-Fos) showed a level of c-Fos similar to that the Lacz-V5-overexpressing group, and the degradation ability was observed only in the recombinant protein (PSMD4-scFv(c-Fos)) that binds to c-Fos (FIG. 10 in A therein). The same results were obtained even when the target protein was GFP-ODC (FIG. 10 in B therein). ProMeD is an affinity-dependent system by which degradation occurs only upon binding between the antibody and the target protein of the recombinant protein, which provides indirect evidence of the target specificity of the technology.

### Example 5: Degradation of Pathogenic Protein by ProMeD

In order for ProMeD to be a platform technology, it should be able to target and degrade various proteins by changing the antibody, and in particular, it should be able to target and degrade pathogenic proteins. To confirm this ability, an antibody that binds to an oncoprotein called BRD4 was used. The antibody is BRD4 scFv.

scFv (BRD4)
(SEQ ID NO: 19)
EVQLLESGGGLVQPGGSLRLSCAASGFTFYGYGMSWVRQAPGKGLEWVSY
IGGYGSSTSYADSVKGRFTISRDNSKNTLYLQMNSLRAEDTAVYYCARGS
AYIDYWGQGTLVTVSSGGGGGGGGSGGGGSDIQMTQSPSSLSASVGDRVT
ITCRASQSISSYLNWYQQKPGKAPKLLIYAASSLQSGVPSRFSGSGSGTD
FTLTISSLQPEDFATYYCQQYNSLYTFGQGTKLEIK
ADRM1-scFv (BRD4)-HTB (755 a.a.)
(SEQ ID NO: 14)
MTTSGALFPSLVPGSRGASNKYLVEFRAGKMSLKGTTVTPDKRKGLVYIQ
QTDDSLIHFCWKDRTSGNVEDDLIIFPDDCEFKRVPQCPSGRVYVLKFKA
GSKRLFFWMQEPKTDQDEEHCRKVNEYLNNPPMPGALGASGSSGHELSAL
GGEGGLQSLLGNMSHSQLMQLIGPAGLGGLGGLGALTGPGLASLLGSSGP
PGSSSSSSSRSQSAAVTPSSTTSSTRATPAPSAPAAASATSPSPAPSSGN
GASTAASPTQPIQLSDLQSILATMNVPAGPAGGQQVDLASVLTPEIMAPI
LANADVQERLLPYLPSGESLPQTADEIQNTLTSPQFQQALGMFSAALASG
QLGPLMCQFGLPAEAVEAANKGDVEAFAKAMQNNAKPEQKEGDTKDKKDE
EEDMSLDLINEVQLLESGGGLVQPGGSLRLSCAASGFTFYGYGMSWVRQA
PGKGLEWVSYIGGYGSSTSYADSVKGRFTISRDNSKNTLYLQMNSLRAED
TAVYYCARGSAYIDYWGQGTLVTVSSGGGGSGGGGSGGGGSDIQMTQSPS
SLSASVGDRVTITCRASQSISSYLNWYQQKPGKAPKLLIYAASSLQSGVP
SRFSGSGSGTDFTLTISSLQPEDFATYYCQQYNSLYTFGQGTKLEIKGAP
HHHHHHDYDIPTTASENLYFQGELGMRGSAGKAGEGEIPAPLAGTVSKIL
VKEGDTVKAGQTVLVLEAMKMETEINAPTDGKVEKVLVKERDAVQGGQGL
IKIGV.

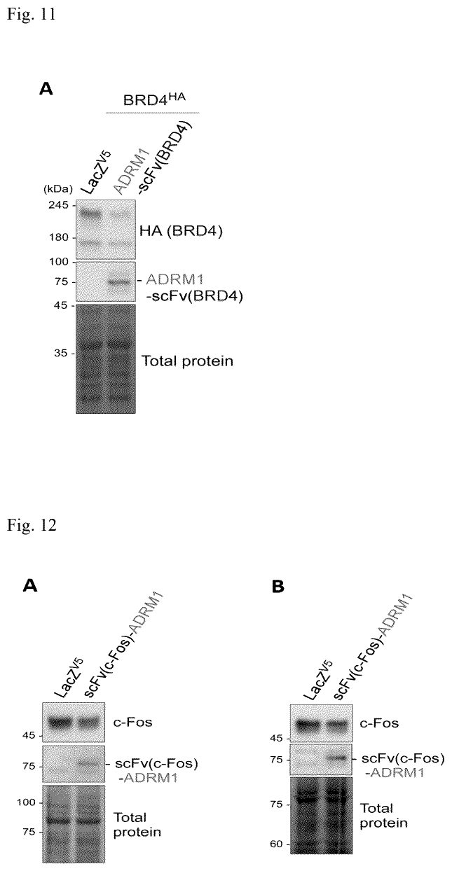

A recombinant protein in which an antibody (scFv(BRD4)) that binds to BRD4 is conjugated to the C-terminus of ADRM1 was constructed by gene cloning. When the recombinant protein was co-expressed with HA-BRD4 in a mammalian cell line, the target protein (HA-BRD4) was effectively degraded compared to that in the control group. Both the long form and the short form simultaneously expressed by splicing exhibited degradation ability (FIG. 11). This result proves that ProMeD can target other oncoproteins in addition to the oncoprotein called c-Fos, implying that it can be an effective platform technology.

### Example 6: Degradation of Endogenous Oncoprotein (c-Fos) by ProMeD System

In order for ProMeD to have therapeutic efficacy, it should be able to degrade endogenous oncoproteins. To confirm this ability, the present inventors used mammalian cell lines MCF-7 and HCT116, which express relatively high levels of c-Fos, which are the cancerous properties of the cells.

The scFv(c-Fos)-ADRM1, which showed the most effectiveness in proof-of-concept experiments to degrade overexpressed target proteins, was transfected into the breast cancer cell line MCF-7. It could be confirmed that the endogenous c-Fos was reduced in the transfected group compared to the control group (FIG. 12 in A therein). Similar results could be obtained even when the same experiment was conducted on the colon cancer cell line HCT116 (FIG. 12 in B therein).

### Example 7: Delivery of ProMeD by Transduction

When a recombinant protein was delivered by transfection, the degradation of endogenous c-Fos was found to be approximately 30%. However, since this methodology could not express the protein in all cells used in the experiment, it could not fully reflect the ability of ProMeD to degrade endogenous c-Fos. To overcome this experimental limitation, transduction was used.

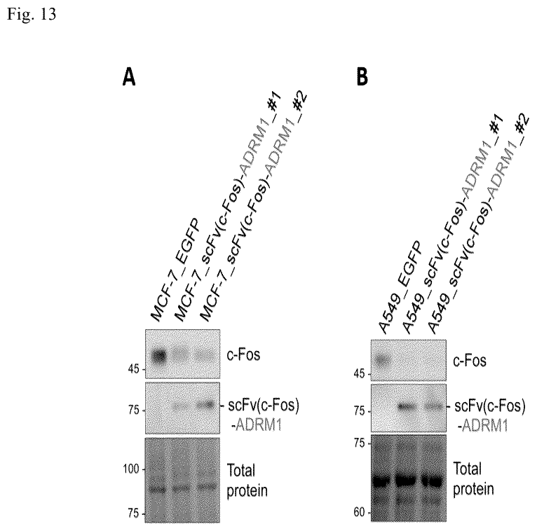

A breast cancer cell line (MCF-7) and lung cancer cell line (A549) stably expressing each of EGFP (a protein that has no effect on the endogenous environment, a control group) and SCFV (c-Fos)-ADRM1 (experimental group) were generated through transduction, selection, and single cell cloning. As a result of comparing the levels of endogenous c-Fos by immunoblotting, it was confirmed that the level of c-Fos in the cell line expressing the recombinant protein was significantly lower than that in the control group (FIG. 13 in A and B therein). This methodology demonstrated the ability of ProMeD to degrade the endogenous target protein because all cells in the experimental group had the recombinant protein (scFv(c-Fos)-ADRM1). These results indicate that the system can degrade oncoproteins present in cells, suggesting that it can have therapeutic efficacy.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

What is claimed is:

1. A fusion protein comprising a ubiquitin receptor and a target-targeting moiety conjugated thereto.

2. The fusion protein according to claim 1, wherein the ubiquitin receptor binds to a proteasome.

3. The fusion protein according to claim 1, wherein the ubiquitin receptor is selected from the group consisting of PSMD2 (Proteasome 26S Subunit Ubiquitin Receptor, Non-ATPase 2), PSMD4 (Proteasome 26S Subunit, Non-ATPase 4), and ADRM1 (Adhesion-regulating molecule 1).

4. The fusion protein according to claim 1, wherein the ubiquitin receptor is selected from the group consisting of SEQ ID NOS: 1 to 3.

5. The fusion protein according to claim 1, wherein the target-targeting moiety is an antibody or an antigen-binding fragment thereof.

6. The fusion protein according to claim 1, wherein the target-targeting moiety is conjugated to an N- or C-terminus of the ubiquitin receptor.

7. The fusion protein according to claim 1, wherein the target-targeting moiety is conjugated directly or via a linker to the ubiquitin receptor.

8. The fusion protein according to claim 7, wherein the linker comprises a rigid linker or a flexible linker.

9. A composition for degrading a target protein, comprising the fusion protein according to claim 1.

10. The composition of claim 9, wherein the composition degrades the target protein by inducing binding of a proteasome to the target protein bound to the fusion protein.

11. An isolated nucleic acid encoding the fusion protein according to claim 1.

12. An expression vector comprising the nucleic acid of claim 11.

13. An isolated host cell comprising the expression vector of claim 12.


SEQUENCE LISTING (19 sequences)

SEQ ID NO: 1 - protein, 908 aa, synthetic construct
  REGION 1..908: note = Synthetic Sequence
MEEGGRDKAPVQPQQSPAAAPGGTDEKPSGKERRDAGDKDKEQELSEEDKQLQDELEMLVERLGEKDTSLYRPALEELRRQIRSSTTSMTSVPKPLKFLRPHYGKLKEIYENMAPGENKRFAADIISVLAMTMSGERECLKYRLVGSQEELASWGHEYVRHLAGEVAKEWQELDDAEKVQREPLLTLVKEIVPYNMAHNAEHEACDLLMEIEQVDMLEKDIDENAYAKVCLYLTSCVNYVPEPENSALLRCALGVFRKFSRFPEALRLALMLNDMELVEDIFTSCKDVVVQKQMAFMLGRHGVFLELSEDVEEYEDLTEIMSNVQLNSNFLALARELDIMEPKVPDDIYKTHLENNRFGGSGSQVDSARMNLASSFVNGFVNAAFGQDKLLTDDGNKWLYKNKDHGMLSAAASLGMILLWDVDGGLTQIDKYLYSSEDYIKSGALLACGIVNSGVRNECDPALALLSDYVLHNSNTMRLGSIFGLGLAYAGSNREDVLTLLLPVMGDSKSSMEVAGVTALACGMIAVGSCNGDVTSTILQTIMEKSETELKDTYARWLPLGLGLNHLGKGEAIEAILAALEVVSEPFRSFANTLVDVCAYAGSGNVLKVQQLLHICSEHFDSKEKEEDKDKKEKKDKDKKEAPADMGAHQGVAVLGIALIAMGEEIGAEMALRTFGHLLRYGEPTLRRAVPLALALISVSNPRLNILDTLSKFSHDADPEVSYNSIFAMGMVGSGTNNARLAAMLRQLAQYHAKDPNNLFMVRLAQGLTHLGKGTLTLCPYHSDRQLMSQVAVAGLLTVLVSFLDVRNIILGKSHYVLYGLVAAMQPRMLVTFDEELRPLPVSVRVGQAVDVVGQAGKPKTITGFQTHTTPVLLAHGERAELATEEFLPVTPILEGFVILRKNPNYDL

SEQ ID NO: 2 - protein, 379 aa, synthetic construct
  REGION 1..379: note = Synthetic Sequence
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEDSDDALLKMTISQQEFGRTGLPDLSSMTEEEQIAYAMQMSLQGAEFGQAESADIDASSAMDTSEPAKEEDDYDVMQDPEFLQSVLENLPGVDPNNEAIRNAMGSLASQATKDGKKDKKEEDKKRP

SEQ ID NO: 3 - protein, 407 aa, synthetic construct
  REGION 1..407: note = Synthetic Sequence
MTTSGALFPSLVPGSRGASNKYLVEFRAGKMSLKGTTVTPDKRKGLVYIQQTDDSLIHFCWKDRTSGNVEDDLIIFPDDCEFKRVPQCPSGRVYVLKFKAGSKRLFFWMQEPKTDQDEEHCRKVNEYLNNPPMPGALGASGSSGHELSALGGEGGLQSLLGNMSHSQLMQLIGPAGLGGLGGLGALTGPGLASLLGSSGPPGSSSSSSSRSQSAAVTPSSTTSSTRATPAPSAPAAASATSPSPAPSSGNGASTAASPTQPIQLSDLQSILATMNVPAGPAGGQQVDLASVLTPEIMAPILANADVQERLLPYLPSGESLPQTADEIQNTLTSPQFQQALGMFSAALASGQLGPLMCQFGLPAEAVEAANKGDVEAFAKAMQNNAKPEQKEGDTKDKKDEEEDMSLD

SEQ ID NO: 4 - protein, 744 aa, synthetic construct
  REGION 1..744: note = Synthetic Sequence
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEDSDDALLKMTISQQEFGRTGLPDLSSMTEEEQIAYAMQMSLQGAEFGQAESADIDASSAMDTSEPAKEEDDYDVMQDPEFLQSVLENLPGVDPNNEAIRNAMGSLASQATKDGKKDKKEEDKKRPKLGTELGSMEVQLQQSGAELVRSGASVKLSCTASGFNIKDYYMHWVKQRPEQGLEWIGWMDPKNGDTEYAPKFQGKATMTADTSSNTAYLQLSSLTSDDTAVYYCHDAITTPTRNFDVWGAGTTVTVSSGGGGSGGGGSGGGGSGGGSDVVMTQTPLSLPVSLGDQASISCRSSQSLVHSNGNTYLHWYLQKPGQSPKLLMYKVSNRFSGVPDRFSGSGSGTDFTLKISRVEAEDLGVYFCSQSTHVPFTFGSGTKLEIKHHHHHHDYDIPTTASENLYFQGELGMRGSAGKAGEGEIPAPLAGTVSKILVKEGDTVKAGQTVLVLEAMKMETEINAPTDGKVEKVLVKERNAVQGGQGLIKIGV

SEQ ID NO: 5 - protein, 759 aa, synthetic construct
  REGION 1..759: note = Synthetic Sequence
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEDSDDALLKMTISQQEFGRTGLPDLSSMTEEEQIAYAMQMSLQGAEFGQAESADIDASSAMDTSEPAKEEDDYDVMQDPEFLQSVLENLPGVDPNNEAIRNAMGSLASQATKDGKKDKKEEDKKRPKLGTELGSMGGGGSGGGGSGGGGSEVQLQQSGAELVRSGASVKLSCTASGFNIKDYYMHWVKQRPEQGLEWIGWMDPKNGDTEYAPKFQGKATMTADTSSNTAYLQLSSLTSDDTAVYYCHDAITTPTRNFDVWGAGTTVTVSSGGGGSGGGGSGGGGSGGGSDVVMTQTPLSLPVSLGDQASISCRSSQSLVHSNGNTYLHWYLQKPGQSPKLLMYKVSNRFSGVPDRFSGSGSGTDFTLKISRVEAEDLGVYFCSQSTHVPFTFGSGTKLEIKHHHHHHDYDIPTTASENLYFQGELGMRGSAGKAGEGEIPAPLAGTVSKILVKEGDTVKAGQTVLVLEAMKMETEINAPTDGKVEKVLVKERNAVQGGQGLIKIGV

SEQ ID NO: 6 - protein, 759 aa, synthetic construct
  REGION 1..759: note = Synthetic Sequence
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEDSDDALLKMTISQQEFGRTGLPDLSSMTEEEQIAYAMQMSLQGAEFGQAESADIDASSAMDTSEPAKEEDDYDVMQDPEFLQSVLENLPGVDPNNEAIRNAMGSLASQATKDGKKDKKEEDKKRPKLGTELGSMEAAAKEAAAKEAAAKEVQLQQSGAELVRSGASVKLSCTASGFNIKDYYMHWVKQRPEQGLEWIGWMDPKNGDTEYAPKFQGKATMTADTSSNTAYLQLSSLTSDDTAVYYCHDAITTPTRNFDVWGAGTTVTVSSGGGGSGGGGSGGGGSGGGSDVVMTQTPLSLPVSLGDQASISCRSSQSLVHSNGNTYLHWYLQKPGQSPKLLMYKVSNRFSGVPDRFSGSGSGTDFTLKISRVEAEDLGVYFCSQSTHVPFTFGSGTKLEIKHHHHHHDYDIPTTASENLYFQGELGMRGSAGKAGEGEIPAPLAGTVSKILVKEGDTVKAGQTVLVLEAMKMETEINAPTDGKVEKVLVKERNAVQGGQGLIKIGV

SEQ ID NO: 7 - protein, 772 aa, synthetic construct
  REGION 1..772: note = Synthetic Sequence
MTTSGALFPSLVPGSRGASNKYLVEFRAGKMSLKGTTVTPDKRKGLVYIQQTDDSLIHFCWKDRTSGNVEDDLIIFPDDCEFKRVPQCPSGRVYVLKFKAGSKRLFFWMQEPKTDQDEEHCRKVNEYLNNPPMPGALGASGSSGHELSALGGEGGLQSLLGNMSHSQLMQLIGPAGLGGLGGLGALTGPGLASLLGSSGPPGSSSSSSSRSQSAAVTPSSTTSSTRATPAPSAPAAASATSPSPAPSSGNGASTAASPTQPIQLSDLQSILATMNVPAGPAGGQQVDLASVLTPEIMAPILANADVQERLLPYLPSGESLPQTADEIQNTLTSPQFQQALGMFSAALASGQLGPLMCQFGLPAEAVEAANKGDVEAFAKAMQNNAKPEQKEGDTKDKKDEEEDMSLDKLGTELGSMEVQLQQSGAELVRSGASVKLSCTASGFNIKDYYMHWVKQRPEQGLEWIGWMDPKNGDTEYAPKFQGKATMTADTSSNTAYLQLSSLTSDDTAVYYCHDAITTPTRNFDVWGAGTTVTVSSGGGGSGGGGSGGGGSGGGSDVVMTQTPLSLPVSLGDQASISCRSSQSLVHSNGNTYLHWYLQKPGQSPKLLMYKVSNRFSGVPDRFSGSGSGTDFTLKISRVEAEDLGVYFCSQSTHVPFTFGSGTKLEIKHHHHHHDYDIPTTASENLYFQGELGMRGSAGKAGEGEIPAPLAGTVSKILVKEGDTVKAGQTVLVLEAMKMETEINAPTDGKVEKVLVKERDAVQGGQGLIKIGV

SEQ ID NO: 8 - protein, 765 aa, synthetic construct
  REGION 1..765: note = Synthetic Sequence
MAGKAGEGEIPAPFAGTVFKILVKEGDTVKAGQTVLVLEAMKMETEINAPTDGKVEKVLVKERDAVQGGQGLIKIGDYDIPTTASENLYFQGELGMRGSHHHHHHEVQLQQSGAELVRSGASVKLSCTASGFNIKDYYMHWVKQRPEQGLEWIGWMDPKNGDTEYAPKFQGKATMTADTSSNTAYLQLSSLTSDDTAVYYCHDAITTPTRNFDVWGAGTTVTVSSGGGGSGGGGSGGGGSGGGSDVVMTQTPLSLPVSLGDQASISCRSSQSLVHSNGNTYLHWYLQKPGQSPKLLMYKVSNRFSGVPDRFSGSGSGTDFTLKISRVEAEDLGVYFCSQSTHVPFTFGSGTKLEIKEFMTTSGALFPSLVPGSRGASNKYLVEFRAGKMSLKGTTVTPDKRKGLVYIQQTDDSLIHFCWKDRTSGNVEDDLIIFPDDCEFKRVPQCPSGRVYVLKFKAGSKRLFFWMQEPKTDQDEEHCRKVNEYLNNPPMPGALGASGSSGHELSALGGEGGLQSLLGNMSHSQLMQLIGPAGLGGLGGLGALTGPGLASLLGSSGPPGSSSSSSSRSQSAAVTPSSTTSSTRATPAPSAPAAASATSPSPAPSSGNGASTAASPTQPIQLSDLQSILATMNVPAGPAGGQQVDLASVLTPEIMAPILANADVQERLLPYLPSGESLPQTADEIQNTLTSPQFQQALGMFSAALASGQLGPLMCQFGLPAEAVEAANKGDVEAFAKAMQNNAKPEQKEGDTKDKKDEEEDMSLD

SEQ ID NO: 9 - protein, 1266 aa, synthetic construct
  REGION 1..1266: note = Synthetic Sequence
MAGKAGEGEIPAPFAGTVFKILVKEGDTVKAGQTVLVLEAMKMETEINAPTDGKVEKVLVKERDAVQGGQGLIKIGDYDIPTTASENLYFQGELGMRGSHHHHHHEVQLQQSGAELVRSGASVKLSCTASGFNIKDYYMHWVKQRPEQGLEWIGWMDPKNGDTEYAPKFQGKATMTADTSSNTAYLQLSSLTSDDTAVYYCHDAITTPTRNFDVWGAGTTVTVSSGGGGSGGGGSGGGGSGGGSDVVMTQTPLSLPVSLGDQASISCRSSQSLVHSNGNTYLHWYLQKPGQSPKLLMYKVSNRFSGVPDRFSGSGSGTDFTLKISRVEAEDLGVYFCSQSTHVPFTFGSGTKLEIKEFMEEGGRDKAPVQPQQSPAAAPGGTDEKPSGKERRDAGDKDKEQELSEEDKQLQDELEMLVERLGEKDTSLYRPALEELRRQIRSSTTSMTSVPKPLKFLRPHYGKLKEIYENMAPGENKRFAADIISVLAMTMSGERECLKYRLVGSQEELASWGHEYVRHLAGEVAKEWQELDDAEKVQREPLLTLVKEIVPYNMAHNAEHEACDLLMEIEQVDMLEKDIDENAYAKVCLYLTSCVNYVPEPENSALLRCALGVFRKFSRFPEALRLALMLNDMELVEDIFTSCKDVVVQKQMAFMLGRHGVFLELSEDVEEYEDLTEIMSNVQLNSNFLALARELDIMEPKVPDDIYKTHLENNRFGGSGSQVDSARMNLASSFVNGFVNAAFGQDKLLTDDGNKWLYKNKDHGMLSAAASLGMILLWDVDGGLTQIDKYLYSSEDYIKSGALLACGIVNSGVRNECDPALALLSDYVLHNSNTMRLGSIFGLGLAYAGSNREDVLTLLLPVMGDSKSSMEVAGVTALACGMIAVGSCNGDVTSTILQTIMEKSETELKDTYARWLPLGLGLNHLGKGEAIEAILAALEVVSEPFRSFANTLVDVCAYAGSGNVLKVQQLLHICSEHFDSKEKEEDKDKKEKKDKDKKEAPADMGAHQGVAVLGIALIAMGEEIGAEMALRTFGHLLRYGEPTLRRAVPLALALISVSNPRLNILDTLSKFSHDADPEVSYNSIFAMGMVGSGTNNARLAAMLRQLAQYHAKDPNNLFMVRLAQGLTHLGKGTLTLCPYHSDRQLMSQVAVAGLLTVLVSFLDVRNIILGKSHYVLYGLVAAMQPRMLVTFDEELRPLPVSVRVGQAVDVVGQAGKPKTITGFQTHTTPVLLAHGERAELATEEFLPVTPILEGFVILRKNPNYDL

SEQ ID NO: 10 - protein, 770 aa, synthetic construct
  REGION 1..770: note = Synthetic Sequence
MAGKAGEGEIPAPFAGTVFKILVKEGDTVKAGQTVLVLEAMKMETEINAPTDGKVEKVLVKERDAVQGGQGLIKIGDYDIPTTASENLYFQGELGMRGSHHHHHHLINEVKLVESGGGLVKPGGSLKLSCAASGFTFSSYGMSWVRQTPEKRLEWVATISRGGSYTYYPDSVKGRFTISRDNAKNTLYLQMSSLRSEDTAIYYCARRETYDEKGFAYWGQGTTLTVSSGGGGSGGGGSGGGGSDIVLTQSPASLTVSLGQRATISCKSSQSLLNSGNQKNYLTWYQQKPGQPPKLLIYWASTRESGIPARFSGSGSGTDFTLNIHPVEEEDAATYYCQNDNSHPLTFGAGTKLEIKRAAAGAPMTTSGALFPSLVPGSRGASNKYLVEFRAGKMSLKGTTVTPDKRKGLVYIQQTDDSLIHFCWKDRTSGNVEDDLIIFPDDCEFKRVPQCPSGRVYVLKFKAGSKRLFFWMQEPKTDQDEEHCRKVNEYLNNPPMPGALGASGSSGHELSALGGEGGLQSLLGNMSHSQLMQLIGPAGLGGLGGLGALTGPGLASLLGSSGPPGSSSSSSSRSQSAAVTPSSTTSSTRATPAPSAPAAASATSPSPAPSSGNGASTAASPTQPIQLSDLQSILATMNVPAGPAGGQQVDLASVLTPEIMAPILANADVQERLLPYLPSGESLPQTADEIQNTLTSPQFQQALGMFSAALASGQLGPLMCQFGLPAEAVEAANKGDVEAFAKAMQNNAKPEQKEGDTKDKKDEEEDMSLD

SEQ ID NO: 11 - protein, 737 aa, synthetic construct
  REGION 1..737: note = Synthetic Sequence
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEDSDDALLKMTISQQEFGRTGLPDLSSMTEEEQIAYAMQMSLQGAEFGQAESADIDASSAMDTSEPAKEEDDYDVMQDPEFLQSVLENLPGVDPNNEAIRNAMGSLASQATKDGKKDKKEEDKKRPLINMAEVKLVESGGGLVKPGGSLKLSCAASGFTFSSFGMHWVRQTPEKRLEWVAYISSGSSTIYYADTVKGRFTISRDNAKNTLYLQMSSLRSEDTAIYYCARSLATAAFAYWGQGTTLTVSSGGGGSGGGGSGGGGSDIVLTQSPASLTVSLGQRATISCRSSQSIVYSNGNTYLEWYQQKPGQPPKLLIYKVSNRFSGIPARFSGSGSGTDFTLNIHPVEEEDAATYYCFQGSHVPYTFGAGTKLEIKGAPHHHHHHDYDIPTTASENLYFQGELGMRGSAGKAGEGEIPAPLAGTVSKILVKEGDTVKAGQTVLVLEAMKMETEINAPTDGKVEKVLVKERDAVQGGQGLIKIGV

SEQ ID NO: 12 - protein, 632 aa, synthetic construct
  REGION 1..632: note = Synthetic Sequence
MAGKAGEGEIPAPFAGTVFKILVKEGDTVKAGQTVLVLEAMKMETEINAPTDGKVEKVLVKERDAVQGGQGLIKIGDYDIPTTASENLYFQGELGMRGSHHHHHHLINVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGAPMTTSGALFPSLVPGSRGASNKYLVEFRAGKMSLKGTTVTPDKRKGLVYIQQTDDSLIHFCWKDRTSGNVEDDLIIFPDDCEFKRVPQCPSGRVYVLKFKAGSKRLFFWMQEPKTDQDEEHCRKVNEYLNNPPMPGALGASGSSGHELSALGGEGGLQSLLGNMSHSQLMQLIGPAGLGGLGGLGALTGPGLASLLGSSGPPGSSSSSSSRSQSAAVTPSSTTSSTRATPAPSAPAAASATSPSPAPSSGNGASTAASPTQPIQLSDLQSILATMNVPAGPAGGQQVDLASVLTPEIMAPILANADVQERLLPYLPSGESLPQTADEIQNTLTSPQFQQALGMFSAALASGQLGPLMCQFGLPAEAVEAANKGDVEAFAKAMQNNAKPEQKEGDTKDKKDEEEDMSLD

SEQ ID NO: 13 - protein, 604 aa, synthetic construct
  REGION 1..604: note = Synthetic Sequence
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEDSDDALLKMTISQQEFGRTGLPDLSSMTEEEQIAYAMQMSLQGAEFGQAESADIDASSAMDTSEPAKEEDDYDVMQDPEFLQSVLENLPGVDPNNEAIRNAMGSLASQATKDGKKDKKEEDKKRPLINVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGAPHHHHHHDYDIPTTASENLYFQGELGMRGSAGKAGEGEIPAPLAGTVSKILVKEGDTVKAGQTVLVLEAMKMETEINAPTDGKVEKVLVKERDAVQGGQGLIKIGV

SEQ ID NO: 14 - protein, 755 aa, synthetic construct
  REGION 1..755: note = Synthetic Sequence
MTTSGALFPSLVPGSRGASNKYLVEFRAGKMSLKGTTVTPDKRKGLVYIQQTDDSLIHFCWKDRTSGNVEDDLIIFPDDCEFKRVPQCPSGRVYVLKFKAGSKRLFFWMQEPKTDQDEEHCRKVNEYLNNPPMPGALGASGSSGHELSALGGEGGLQSLLGNMSHSQLMQLIGPAGLGGLGGLGALTGPGLASLLGSSGPPGSSSSSSSRSQSAAVTPSSTTSSTRATPAPSAPAAASATSPSPAPSSGNGASTAASPTQPIQLSDLQSILATMNVPAGPAGGQQVDLASVLTPEIMAPILANADVQERLLPYLPSGESLPQTADEIQNTLTSPQFQQALGMFSAALASGQLGPLMCQFGLPAEAVEAANKGDVEAFAKAMQNNAKPEQKEGDTKDKKDEEEDMSLDLINEVQLLESGGGLVQPGGSLRLSCAASGFTFYGYGMSWVRQAPGKGLEWVSYIGGYGSSTSYADSVKGRFTISRDNSKNTLYLQMNSLRAEDTAVYYCARGSAYIDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQYNSLYTFGQGTKLEIKGAPHHHHHHDYDIPTTASENLYFQGELGMRGSAGKAGEGEIPAPLAGTVSKILVKEGDTVKAGQTVLVLEAMKMETEINAPTDGKVEKVLVKERDAVQGGQGLIKIGV

SEQ ID NO: 15 - protein, 251 aa, synthetic construct
  REGION 1..251: note = Synthetic Sequence
EVQLQQSGAELVRSGASVKLSCTASGFNIKDYYMHWVKQRPEQGLEWIGWMDPKNGDTEYAPKFQGKATMTADTSSNTAYLQLSSLTSDDTAVYYCHDAITTPTRNFDVWGAGTTVTVSSGGGGSGGGGSGGGGSGGGSDVVMTQTPLSLPVSLGDQASISCRSSQSLVHSNGNTYLHWYLQKPGQSPKLLMYKVSNRFSGVPDRFSGSGSGTDFTLKISRVEAEDLGVYFCSQSTHVPFTFGSGTKLEIK

SEQ ID NO: 16 - protein, 248 aa, synthetic construct
  REGION 1..248: note = Synthetic Sequence
EVKLVESGGGLVKPGGSLKLSCAASGFTFSSYGMSWVRQTPEKRLEWVATISRGGSYTYYPDSVKGRFTISRDNAKNTLYLQMSSLRSEDTAIYYCARRETYDEKGFAYWGQGTTLTVSSGGGGSGGGGSGGGGSDIVLTQSPASLTVSLGQRATISCKSSQSLLNSGNQKNYLTWYQQKPGQPPKLLIYWASTRESGIPARFSGSGSGTDFTLNIHPVEEEDAATYYCQNDNSHPLTFGAGTKLEIK

SEQ ID NO: 17 - protein, 248 aa, synthetic construct
  REGION 1..248: note = Synthetic Sequence
EVKLVESGGGLVKPGGSLKLSCAASGFTFSSFGMHWVRQTPEKRLEWVAYISSGSSTIYYADTVKGRFTISRDNAKNTLYLQMSSLRSEDTAIYYCARSLATAAFAYWGQGTTLTVSSGGGGSGGGGSGGGGSDIVLTQSPASLTVSLGQRATISCRSSQSIVYSNGNTYLEWYQQKPGQPPKLLIYKVSNRFSGIPARFSGSGSGTDFTLNIHPVEEEDAATYYCFQGSHVPYTFGAGTKLEIKGAP

SEQ ID NO: 18 - protein, 114 aa, synthetic construct
  REGION 1..114: note = Synthetic Sequence
VQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 19 - protein, 237 aa, synthetic construct
  REGION 1..237: note = Synthetic Sequence
EVQLLESGGGLVQPGGSLRLSCAASGFTFYGYGMSWVRQAPGKGLEWVSYIGGYGSSTSYADSVKGRFTISRDNSKNTLYLQMNSLRAEDTAVYYCARGSAYIDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQYNSLYTFGQGTKLEIK